(19) United States
(12) Patent Application Publication
(10) Pub. No.: US 2012/0172313 A1
(43) Pub. Date: Jul. 5, 2012

(54) TREATMENT OF PROLIFERATIVE DISEASES

(76) Inventors: MARK DARREN HULETT, ELTHAM (AU); IVAN KA HO POON, KEW (AU); AMY ALEXANDRA BAXTER, BRUNSWICK (AU); MARILYN ANDERSON, KEILOR (AU); FUNG LAY, RESERVOIR (AU)

(21) Appl. No.: 13/166,960

(22) Filed: Jun. 23, 2011

Related U.S. Application Data
(60) Provisional application No. 61/358,126, filed on Jun. 24, 2010.

(51) Int. Cl. A61K 38/16; C07K 14/415; C12Q 1/18; A61P 35/00; A61P 35/02

(52) U.S. Cl. 514/19.4; 514/19.3; 514/19.6; 514/19.5; 435/32; 530/324; 530/350

(57) ABSTRACT

The present invention relates to methods for preventing or treating proliferative diseases. In particular, the present invention relates to the use of compositions derived or derivable from plants, such as plant defensins, particularly in methods for the prevention or treatment of proliferative diseases such as cancer. The present invention also relates to associated uses, systems and kits.

### CROSS-REFERENCE TO RELATED APPLICATIONS

This application claims priority to U.S. Provisional Patent Application No. 61/358,126 filed on Jun. 24, 2010, which is hereby incorporated by reference in its entirety.

### FIELD OF THE INVENTION

The present invention relates to methods for preventing or treating proliferative diseases. In particular, the present invention relates to the use of compositions derived or derivable from plants, such as plant defensins, particularly in methods for the prevention or treatment of proliferative diseases such as cancer. The present invention also relates to associated uses, systems and kits.

### STATEMENT REGARDING FEDERALLY FUNDED RESEARCH

Not applicable.

### BACKGROUND TO THE INVENTION

Plants are known to produce a variety of chemical compounds, either constitutively or inducibly, to protect themselves against environmental stresses, wounding, or microbial invasion.

Of the plant antimicrobial proteins that have been characterized to date, a large proportion share common characteristics. They are generally small (<10 kDa), highly basic proteins and often contain an even number of cysteine residues (typically 4, 6 or 8). These cysteines all participate in intramolecular disulfide bonds and provide the protein with structural and thermodynamic stability (Broekaert et al. (1997)). Based on amino acid sequence identities, primarily with reference to the number and spacing of the cysteine residues, a number of distinct families have been defined. They include the plant defensins (Broekaert et al, 1995, 1997; Lay et al., 2003a), thionins (Bohlmann, 1994), lipid transfer proteins (Kader, 1996, 1997), hevein (Broekaert et al., 1992) and knottin-type proteins (Cammue et al., 1992), as well as antimicrobial proteins from Macadamia integrifolia (Marcus et al., 1997; McManus et al., 1999) and Impatiens balsamina (Tailor et al., 1997; Patel et al., 1998) (Table 1). All these antimicrobial proteins appear to exert their activities at the level of the plasma membrane of the target microorganisms, although it is likely that the different protein families act via different mechanisms (Broekaert et al., 1997). The cyclotides are a new family of small, cysteine-rich plant peptides that are common in members of the Rubiaceae and Violaceae families (reviewed in Craik et al., 1999, 2004; Craik, 2001). These unusual cyclic peptides (Table 1) have, been ascribed various biological activities including antibacterial (Tam, et al., 1999), anti-HIV (Gustafson et al., 1994) and insecticidal (Jennings et al., 2001) properties.

TABLE 1
Small, cysteine-rich antimicrobial proteins in plants.

| | | No. | |
|---|---|---|---|
| | Repre- | of | |
| Peptide | sentative | amino | Consensus |
| Family | member | acids | sequence |
| Plant defensins | Rs-AFP2 | 51 | 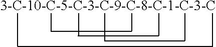 |
| α/β- Thionin (8-Cys type) | α- Purothionin | 45 | 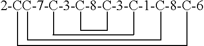 |
| Lipid transfer protein | Ace-AMP1 | 93 | 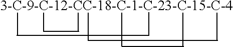 |
| Hevein- type | Ac-AMP2 | 30 | 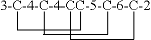 |
| Knottin- type | Mj-AMP1 | 36 | 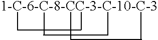 |
| Macada- mia | MiAMP1 | 76 | 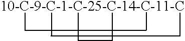 |
| Impatiens | Ib-AMP1 | 20 | 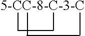 |
| Cyclotide | Kalata B1 | 29 | 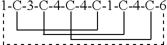 |

The size of the mature protein and spacing of cysteine residues for representative members of plant antimicrobial proteins is shown in Table 1. The numbers in the consensus sequence represent the number of amino acids between the highly conserved cysteine residues in the representative member but other members of the family may vary slightly in the inter-cysteine lengths. The disulfide connectivities are given by connecting lines. The cyclic backbone of the cyclotides is depicted by the broken line (from Lay and Anderson, 2005).

### Defensins

The term “defensin” has previously been used in the art to describe a diverse family of molecules that are produced by many different species and which function in innate defense against pathogens including bacteria, fungi, yeast and viruses.

### Plant Defensins

Plant defensins (also termed γ-thionins) are small (˜5 kDa, 45 to 54 amino acids), basic proteins with eight cysteine residues that form four strictly conserved disulfide bonds with a Cys_{I}-Cys_{VIII}, Cys_{II}-Cys_{IV}, Cys_{III}-Cys_{VI} and Cys_{V}-Cys_{VII} configuration. As well as these four strictly conserved disulfide bonds, some plant defensins have an additional disulfide bond (Lay et al., 2003a, 2003b; Janssen et al., 2003).

The name “plant defensin” was coined in 1995 by Terras and colleagues who isolated two antifungal proteins from radish seeds (Rs-AFP1 and Rs-AFP2) and noted that at a primary and three-dimensional structural level these proteins were distinct from the plant α-/β-thionins but shared some structural similarities to insect and mammalian defensins (Terras et al., 1995; Broekaert et al., 1995).

Plant defensins exhibit clear, although relatively limited, sequence conservation. Strictly conserved are the eight cysteine residues and a glycine at position 34 (numbering relative to Rs-AFP2). In most of the sequences, a serine at position 8, an aromatic residue at position 11, a glycine at position 13 and a glutamic acid at position 29 are also conserved (Lay et al., 2003a; Lay and Anderson, 2005).

The three-dimensional solution structures of the first plant defensins were elucidated in 1993 by Bruix and colleagues for γ1-P and γ1-H. Since that time, the structures of other seed-derived and two flower-derived (NaD1 and PhD1) defensins have been determined (Lay et al., 2003b; Janssen et al., 2003). All these defensins elaborate a motif known as the cysteine-stabilized αβ (CSαβ) fold and share highly superimposable three-dimensional structures that comprise a well-defined α-helix and a triple-stranded antiparallel β-sheet. These elements are organized in a βαββ arrangement and are reinforced by four disulfide bridges.

The CSαβ motif is also displayed by insect defensins and scorpion toxins. In comparing the amino acid sequences of the structurally characterized plant defensins, insect defensins and scorpion toxins, it is apparent that the CSαβ scaffold is highly permissive to size and compositional differences.

The plant defensin/γ-thionin structure contrasts to that which is adopted by the α- and β-thionins. The α- and β-thionins form compact, amphipathic, L-shaped molecules where the long vertical arm of the L is composed of two α-helices, and the short arm is formed by two antiparallel β-strands and the last (˜10) C-terminal residues. These proteins are also stabilized by three or four disulfide bonds (Bohlmann and Apel, 1991).

Plant defensins have a widespread distribution throughout the plant kingdom and are likely to be present in most, if not all, plants. Most plant defensins have been isolated from seeds where they are abundant and have been characterized at the molecular, biochemical and structural levels (Broekaert et al., 1995; Thomma et al., 2003; Lay and Anderson, 2005). Defensins have also been identified in other tissues including leaves, pods, tubers, fruit, roots, bark and floral tissues (Lay and Anderson, 2005).

An amino acid sequence alignment of several defensins that have been identified, either as purified protein or deduced from cDNAs, has been published by Lay and Anderson (2005). Other plant defensins have been disclosed in U.S. Pat. No. 6,911,577, International Patent Publication No. WO 00/11196 and International Patent Publication No. WO 00/68405, the entire contents of which are incorporated herein by reference.

### Mammalian Defensins

The mammalian defensins form three distinct structural subfamilies known as the α-, β- and θ-defensins. In contrast to the plant defensins, all three subfamilies contain only six cysteine residues which differ with respect to their size, the placement and connectivity of their cysteines, the nature of their precursors and their sites of expression (Selsted et al., 1993; Hancock and Lehrer, 1998; Tang et al., 1999a, b; Lehrer and Ganz, 2002). All subfamilies have an implicated role in innate host immunity and more recently, have been linked with adaptive immunity as immunostimulating agents (Tang et al., 1999b; Lehrer and Ganz, 2002). It was in the context of their defense role that the name “defensin” was originally coined (Ganz et al., 1985; Selsted et al., 1985).

The α-defensins (also known as classical defensins) are 29-35 amino acids in length and their six cysteine residues form three disulfide bonds with a Cys_{I}-Cys_{VI}, Cys_{II}-Cys_{IV} and Cys_{III}-Cys_{V} configuration (Table 2).

In contrast to the α-defensins, the β-defensins are larger (36-42 amino acids in size) and have a different cysteine pairing (Cys_{I}-Cys_{V}, Cys_{II}-Cys_{IV} and Cys_{III}-Cys_{VI}) and spacing (Tang and Selsted, 1993). They are also produced as preprodefensins. However, their prodomains are much shorter. Analogous to the α-defensins, the synthesis of β-defensins can be constitutive or can be induced following injury or exposure to bacteria, parasitic protozoa, bacterial lipopolysaccharides, and also in response to humoral mediators (i.e. cytokines) (Diamond et al., 1996; Russell et al., 1996; Tarver et al., 1998).

The size of the mature protein and spacing of cysteine residues for representative members of defensin and defensin-like proteins from insects and mammals is shown in Table 2. The numbers in the consensus sequence represent the number of amino acids between the highly conserved cysteine residues in the representative member, but other members of the family may vary slightly in the inter-cysteine lengths. The disulfide connectivities are given by connecting lines. The cyclic backbone of the mammalian theta-defensins is depicted by the broken line.

TABLE 2
Representative members of defensin and defensin-like proteins from insects and mammals

| | Representative | No. of | | |
|---|---|---|---|---|
| Peptide family | member | amino acids | Consensus sequence | Reference |
| Insect defensin-like | Drosomycin | 44 | 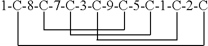 | Lamberty et al., 2001 |
| Insect defensin | Insect defensin A | 40 | 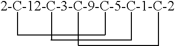 | Cornet et al., 1995 |
| Mammalian α-defensin | HNP-4 | 34 | 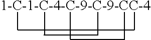 | Harwig et al., 1992 |
| Mammalian β-defensin | HBD-1 | 36 | 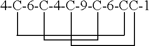 | Bensch et al., 1995 |
| Mammalian θ-defensin | RTD-1 | 18 | 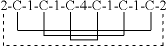 | Tang et al., 1999a, b Trabi et al., 2001 |

### Insect Defensins

A large number of defensin and defensin-like proteins have been identified in insects. These proteins are produced in the fat body (equivalent of the mammali an liver) from which they are subsequently released into the hemolymph (Lamberty et al., 1999). Most insect defensins have three disulfide bonds. However, a number of related proteins, namely drosomycin from Drosophila melanogaster, have four disulfides (Fehlbaum et al., 1994; Landon et al., 1997) (Table 2).

The three-dimensional structures of several insect defensins have been solved (e.g. Hanzawa et al., 1990; Bonmatin et al., 1992; Cornet et al., 1995; Lamberty et al., 2001; Da Silva et al., 2003). Their global fold, as typified by insect defensin A, features an α-helix, a double-stranded antiparallel β-sheet and a long N-terminal loop. These elements of secondary structure are stabilized by three disulfide bonds that are arranged in a Cy_{sI}-Cy_{sIV}, Cy_{sII}-Cy_{sV} and Cy_{sIII}-Cy_{sVI} configuration (Bonmatin et al., 1992; Cornet et al., 1995).

### Two Classes of Plant Defensins

Plant defensins can be divided into two major classes according to the structure of the precursor proteins predicted from cDNA clones (Lay et al., 2003a) (FIG. 8). In the first and largest class, the precursor protein is composed of an endoplasmic reticulum (ER) signal sequence and a mature defensin domain. These proteins enter the secretory pathway and have no obvious signals for post-translational modification or subcellular targeting (FIG. 8A).

The second class of defensins are produced as larger precursors with C-terminal prodomains or propeptides (CTPPs) of about 33 amino acids (FIG. 8B). Class II defensins have been identified in solanaceous species where they are expressed constitutively in floral tissues (Lay et al., 2003a; Gu et al., 1992; Milligan et al., 1995; Brandstadter et al., 1996) and fruit (Aluru et al., 1999) and in salt stressed leaves (Komori et al., 1997; Yamada et al., 1997). The CTPP of the solanaceous defensins from Nicotiana alata (NaD1) and Petunia hybrida (PhD1 and PhD2) is removed proteolytically during maturation (Lay et al., 2003a).

The CTPPs on the solanaceous defensins have an unusually high content of acidic and hydrophobic amino acids. Interestingly, at neutral pH, the negative charge of the CTPP counter-balances the positive charge of the defensin domain (Lay and Anderson, 2005).

### Biological Activity of Plant Defensins

Some biological activities have been attributed to plant defensins including growth inhibitory effects on fungi (Broekaert et al., 1997; Lay et al., 2003a; Osborn et al., 1995; Terras et al., 1993), and Gram-positive and Gram-negative bacteria (Segura et al., 1998; Moreno et al., 1994; Zhang and Lewis, 1997). Some defensins are also effective inhibitors of digestive enzymes such as α-amylases (Zhang et al., 1997; Bloch et al., 1991) and serine proteinases (Wijaya et al., 2000; Melo et al., 2002), two functions consistent with a role in protection against insect herbivory. This is supported by the observation that bacterially expressed mung bean defensin, VrCRP, is lethal to the bruchid Callosobruchus chinensis when incorporated into an artificial diet at 0.2% (w/w) (Chen et al., 2002). Some defensins also inhibit protein translation (Mendez et al., 1990; Colilla et al., 1990; Mendez et al., 1996) or bind to ion channels (Kushmerick et al., 1998). A defensin from Arabidopsis halleri also confers zinc tolerance, suggesting a role in stress adaptation (Mirouze et al., 2006). More recently, a sunflower defensin was shown to induce cell death in Orobanche parasite plants (de Zélicourt et al., 2007).

### Antifungal Activity

The best characterized activity of some but not all plant defensins is their ability to inhibit, with varying potencies, a large number of fungal species (for examples, see Broekaert et al., 1997; Lay et al., 2003a; Osborn et al., 1995). Rs-AFP2, for example, inhibits the growth of Phoma betae at 1 μg/mL, but is ineffective against Sclerotinia sclerotiorum at 100 μg/mL (Terras et al., 1992). Based on their effects on the growth and morphology of the fungus, Fusarium culmorum, two groups of defensins can be distinguished. The “morphogenic” plant defensins cause reduced hyphal elongation with a concomitant increase in hyphal branching, whereas the “non-morphogenic” plant defensins reduce the rate of hyphal elongation, but do not induce marked morphological distortions (Osborn et al., 1995).

More recently, the pea defensin Psd1 has been shown to be taken up intracellularly and enter the nuclei of Neurospora crassa where it interacts with a nuclear cyclin-like protein involved in cell cycle control (Lobo et al., 2007). For MsDef1, a defensin from alfalfa, two mitogen-activated protein (MAP) kinase signalling cascades have a major role in regulating MsDef1 activity on Fusarium graminearum (Ramamoorthy et al., 2007).

Permeabilization of fungal membranes has also been reported for some plant defensins (Lay and Anderson, 2005). For example, NaD1 is a plant defensin isolated from floral tissue of Nicotiana alata. The amino acid and coding sequences of NaD1 are disclosed in International Patent Publication No. WO 02/063011, the entire contents of which are incorporated by reference herein. NaD1 was tested in vitro for antifungal activity against the filamentous fungi Fusarium oxysporum f. sp. vasinfectum (Fov), Verticillium dahliae, Thielaviopsis basicola, Aspergillus nidulans and Leptosphaeria maculans. At 1 μM, NaD1 retarded the growth of Fov and L. maculans by 50% while V. dahliae, T. basicola, and A. nidulans were all inhibited by approximately 65%. At 5 μM NaD1, the growth of all five species was inhibited by more than 80%. These five fungal species are all members of the ascomycete phylum and are distributed among three classes in the subphylum pezizomycotiria. These fungi are agronomically important fungal pathogens. All filamentous fungi tested thus far are sensitive to inhibition by NaD1 (van der Weerden et al., 2008).

The importance of the four disulfide bonds in NaD1 was investigated by reducing and alkylating the cysteine residues. Reduced and alkylated NaD1 (NaD1_{R&A}) was completely inactive in the growth inhibitory assays with Fov, even at a concentration ten-fold higher than the IC₅₀ for NaD1 (van der Weerden et al., 2008).

Prior Work with Antimicrobial Peptides and Tumour Cells

### Use of Small Cysteine-Rich/Cationic Antimicrobial Peptides in the Treatment of Human Disease

There is an increasing body of literature implicating human α- and β-defensins in various aspects of cancer, tumourigenesis, angiogenesis and invasion. The use of mammalian defensins has also been proposed for the treatment of viral and fungal infections and as an alternative or adjunct to antibiotic treatment of bacterial infections. However, their cytotoxicity towards mammalian cells remains a significant barrier. Moss et al (U.S. Pat. No. 7,511,015) have shown that modification of the defensin peptide through ribosylation or ADP-ribosylation of arginine residues modifies the toxicity of the peptide and enhances its antimicrobial properties.

The review by Mader and Hoskin (2006) describes the use of cationic antimicrobial peptides as novel cytotoxic agents for cancer treatment. It should be noted however that a review by Pelegrini and Franco (2005) incorrectly describes α-/β-thionins from mistletoe, which are anticancer molecules, as γ-thionihs (another name for plant defensins). The person skilled in the art would understand that such prior art does not relate to plant defensins (γ-thionins) but instead to the structurally and functionally distinct α-/β-thionins.

Reports of Plant Defensins with Antiproliferative Activity on Human Cancer Cells

Since 2004, some isolated reports have suggested that plant defensin(-like) proteins could also display in vitro antiproliferative activity against various human tumour cell lines (with differing potencies) (see, for example, Wong and Ng (2005), Ngai and Ng (2005), Ma et al. (2009) and Lin et al. (2009)). These proteins have largely been isolated from leguminous plants (e.g. beans). The assignment of these proteins to the plant defensin class was based on their estimated molecular mass (˜5 kDa) and in some cases, on limited N-terminal amino acid similarities to known defensin sequences. However, the proteins as disclosed in these references lack the strictly conserved cysteine residues and cysteine spacings that define defensins. In addition, the proteins disclosed in such references are not Class II defensins, nor are they from the family Solanaceae.

A review of the literature indicates that the Capsicum chinese defensin (CcD1) is the only other Class II defensin of the Solanaceae family that has been previously implicated as having the potential to inhibit the viability of mammalian cells (Anaya-Lopez et al., 2006). It is reported that the transfection of an expression construct encoding a full-length sequence for CcD1 into the bovine endothelial cell line BE-E6E7 resulted in conditioned media that exhibited anti-proliferative effects on the human transformed cell line HeLa. There are a number of major flaws in the experimental design and interpretation of these data that make it impossible for the person skilled in the art to draw a valid conclusion from the described studies as to whether CcD1 exhibits anti-proliferative activity. These include: (i) although mRNA for CcD1 was suggested in the transfected cells, no evidence was provided to demonstrate that the CcD1 protein was actually expressed in the conditioned media, (ii) the use of the full-length open-reading frame of CcD1 rather than the mature coding domain would require the processing of the expressed precursor by removal of the CTPP domain to produce an “active” defensin—this was not demonstrated, (iii) the process of transfection can result in changes to a cell and the control for the transfection experiment was not adequate in that untransfected cells were used rather than the correct control of vector alone transfected cells, (iv) the use of conditioned media rather than purified CcD1 protein could influence the experimental readout as components of the media or other secreted molecules from the transfected cells may themselves, or in combination with CcD1, have anti-proliferative activity, (v) the expression levels of CcD1 mRNA in the various transfected endothelial cell populations (Anaya-Lopez et al., 2006, FIG. 2) do not correlate with the proposed anti-proliferative activity of the CcD1 transfected cell conditioned media (Anaya-Lopez et al., 2006, FIG. 4) as there is no statistically significant difference between the observed anti-proliferative responses mediated by the different conditioned media samples. It should also be noted that these deficiencies in the experimental design and interpretation were expressly acknowledged in an independently published paper by the same authors in 2008 (Loenza-Angeles et al., 2008). Based on these observations, it would be impossible for the person skilled in the art to interpret from Anaya-Lopez et al. (2006) that CcD1 has any anti-proliferative activity against mammalian cells.

The inventors have previously disclosed in International Patent Publication No. WO 02/063011 certain novel defensins and their use in inducing resistance in plants or parts of plants to pathogen infestation. The entire contents of WO 02/063011 are incorporated herein by reference.

As a result of further studies into plant defensins, it has surprisingly been determined that Class II defensins from the Solanaceae plant family have potent cytotoxic properties. These significant findings therefore describe a novel and important way in which proliferative diseases may be prevented and treated. Accordingly, these findings provide for methods for the prevention and treatment of proliferative diseases such as cancer, as well as associated systems and kits.

### SUMMARY OF THE INVENTION

In a first aspect of the present invention, there is provided a plant defensin for use in preventing or treating a proliferative disease.

In a second aspect of the present invention, there is provided a nucleic acid encoding the plant defensin of the first aspect.

In a third aspect of the present invention, there is provided a vector comprising the nucleic acid of the second aspect.

In a fourth aspect of the present invention, there is provided a host cell comprising the vector of the third aspect.

In a fifth aspect of the present invention, there is provided an expression product produced by the host cell of the fourth aspect.

In a sixth aspect of the present invention, there is provided a pharmaceutical composition for use in preventing or treating a proliferative disease, wherein the pharmaceutical composition comprises the plant defensin of the first aspect, the nucleic acid of the second aspect, the vector of the third aspect, the host cell of the fourth aspect or the expression product of the fifth aspect, together with a pharmaceutically acceptable carrier, diluent or excipient.

In a seventh aspect of the present invention, there is provided a method for preventing or treating a proliferative disease, wherein the method comprises administering to a subject a therapeutically effective amount of the plant defensin of the first aspect, the nucleic acid of the second aspect, the vector of the third aspect, the host cell of the fourth aspect, the expression product of the fifth aspect or the pharmaceutical composition of the sixth aspect, thereby preventing or treating the proliferative disease.

In an eighth aspect of the present invention, there is provided use of the plant defensin of the first aspect, the nucleic acid of the second aspect, the vector of the third aspect, the host cell of the fourth aspect, the expression product of the fifth aspect or the pharmaceutical composition of the sixth aspect in the preparation of a medicament for preventing or treating a proliferative disease.

In a ninth aspect of the present invention, there is provided a kit for preventing or treating a proliferative disease, wherein the kit comprises a therapeutically effective amount of the plant defensin of the first aspect, the nucleic acid of the second aspect, the vector of the third aspect, the host cell of the fourth aspect, the expression product of the fifth aspect or the pharmaceutical composition of the sixth aspect.

In a tenth aspect of the present invention, there is provided use of the kit of the ninth aspect for preventing or treating a proliferative disease, wherein the therapeutically effective amount of the plant defensin of, the first aspect, the nucleic acid of the second aspect, the vector of the third aspect, the host cell of the fourth aspect, the expression product of the fifth aspect or the pharmaceutical composition of the sixth aspect is administered to a subject, thereby preventing or treating the proliferative disease.

In an eleventh aspect of the present invention, there is provided a method for screening for cytotoxicity of plant defensins against mammalian tumour cells, wherein the method comprises contacting the plant defensin of the first aspect, the nucleic acid of the second aspect, the vector of the third aspect, the host cell of the fourth aspect, the expression product of the fifth aspect or the pharmaceutical composition of the sixth aspect with a mammalian cell line, and assaying for cytoxicity against the mammalian cell line due to contact with the plant defensin.

In a twelfth aspect of the present invention, there is provided a plant defensin screened by the method of the eleventh aspect.

In a thirteenth aspect of the present invention, there is provided a method for producing a plant defensin with reduced haemolytic activity, wherein the method comprises introducing into the plant defensin at least one alanine residue at or near the N-terminal of the defensin.

In a fourteenth aspect of the present invention, there is provided a plant defensin with reduced haemolytic activity produced by the method according to the thirteenth aspect.

### DEFINITIONS

The term “derivable” includes, and may be used interchangeably with, the terms “obtainable” and “isolatable”. Compositions or other matter of the present invention that is “derivable”, “obtainable” or “isolatable” from a particular source or process include not only compositions or other matter derived, obtained or isolated from that source or process, but also the same compositions or matter however sourced or produced.

As used herein the term “polypeptide” means a polymer made up of amino acids linked together by peptide bonds, and includes fragments or analogues thereof. The terms “polypeptide”, “protein” and “amino acid” are used interchangeably herein, although for the purposes of the present invention a “polypeptide” may constitute a portion of a full length protein.

The term “nucleic acid” as used herein refers to a single- or double-stranded polymer of deoxyribonucleotide, ribonucleotide bases or known analogues of natural nucleotides, or mixtures thereof. The term includes reference to the specified sequence as well as to the sequence complementary thereto, unless otherwise indicated. The terms “nucleic acid”, “polynucleotide” and “nucleotide sequence” are used herein interchangeably. It will be understood that “5′ end” as used herein in relation to a nucleic acid corresponds to the N-terminus of the encoded polypeptide and “3′ end” corresponds to the C-terminus of the encoded polypeptide.

The term “purified” means that the material in question has been removed from its natural environment or host, and associated impurities reduced or eliminated such that the molecule in question is the predominant species present. The term “purified” therefore means that an object species is the predominant species present (ie., on a molar basis it is more abundant than any other individual species in the composition), and preferably a substantially purified fraction is a composition wherein the object species comprises at least about 30 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species. The terms “purified” and “isolated” may be used interchangeably. Purity and homogeneity are typically determined using analytical chemistry techniques such as polyacrylamide gel electrophoresis or high performance liquid chromatography. A protein or nucleic acid that is the predominant species present in a preparation is substantially purified. The term “purified” in some embodiments denotes that a protein or nucleic acid gives rise to essentially one band in an electrophoretic gel.

The term “fragment” refers to a polypeptide or nucleic acid that encodes a constituent or is a constituent of a polypeptide or nucleic acid of the invention thereof. Typically the fragment possesses qualitative biological activity in common with the polypeptide or nucleic acid of which it is a constituent. A peptide fragment may be between about 5 to about 150 amino acids in length, between about 5 to about 100 amino acids in length, between about 5 to about 50 amino acids in length, or between about 5 to about 25 amino acids in length. Alternatively, the peptide fragment may be between about 5 to about 15 amino acids in length. The term “fragment” therefore includes a polypeptide that is a constituent of a full-length plant defensin polypeptide and possesses qualitative biological activity in common with a full-length plant defensin polypeptide. A fragment may be derived from a full-length plant defensin polypeptide or alternatively may be synthesised by some other means, for example chemical synthesis.

The term “fragment” may also refer to a nucleic acid that encodes a constituent or is a constituent of a polynucleotide of the invention. Fragments of a nucleic acid do not necessarily need to encode polypeptides which retain biological activity. Rather the fragment may, for example, be useful as a hybridization probe or PCR primer. The fragment may be derived from a polynucleotide of the invention or alternatively may be synthesized by some other means, for example chemical synthesis. Nucleic acids of the present invention and fragments thereof may also be used in the production of antisense molecules using techniques known to those skilled in the art.

The term “recombinant” when used with reference, for example, to a cell, nucleic acid, protein or vector, indicates that the cell, nucleic acid, protein or vector has been modified by the introduction of a heterologous nucleic acid or protein or by the alteration of a native nucleic acid or protein, or that the cell is derived from a cell so modified. Accordingly, “recombinant” cells express genes that are not found within the native (non-recombinant) form of the cell or express native genes that are otherwise abnormally expressed, under expressed or not expressed at all. By the term “recombinant nucleic acid” is meant a nucleic acid, originally formed in vitro, in general, by the manipulation of a nucleic acid, for example, using polymerases and endonucleases, in a form not normally found in nature. In this manner, operable linkage of different sequences is achieved. Thus an isolated nucleic acid, in a linear form, or an expression vector formed in vitro by ligating DNA molecules that are not normally joined, are both considered “recombinant” for the purposes of this invention. It is understood that once a recombinant nucleic acid is made and reintroduced into a host cell or organism, it will replicate non-recombinantly, i.e., using the in vivo cellular machinery of the host cell rather than in vitro manipulations. However, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The terms “identical” or percent “identity” in the context of two or more polypeptide (or nucleic acid) sequences, refer to two or more sequences or sub-sequences that are the same or have a specified percentage of amino acid residues (or nucleotides) that are the same over a specified region, when compared and aligned for maximum correspondence over a comparison window or designated region, as measured using sequence comparison algorithms, or by manual alignment and visual inspection, such techniques being well known to the person skilled in the art.

As used herein the term “treatment”, refers to any and all uses which remedy a disease state or symptoms, prevent the establishment of disease, or otherwise prevent, hinder, retard, ameliorate or reverse the progression of disease or other undesirable symptoms in any way whatsoever.

Unless defined otherwise, all technical and scientific terms used herein have the same meaning as commonly understood by one of ordinary skill in the art (e.g. in cell biology, chemistry, molecular biology and cell culture). Standard techniques used for molecular and biochemical methods can be found in Sambrook et al., Molecular Cloning: A Laboratory Manual, 3^{rd} ed. (2001) Cold Spring Harbor Laboratory Press, Cold Spring Harbor, N.Y. and Ausubel et al., Short Protocols in Molecular Biology (1999) 4^{th} Ed, John Wiley & Sons, Inca—and the full version entitled Current Protocols in Molecular Biology).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, reference to numerical values, unless stated otherwise, is to be taken as meaning “about” that numerical value. The term “about” is used to indicate that a value includes the inherent variation of error for the device and the method being employed to determine the value, or the variation that exists among the study subjects.

The reference to any prior art in this specification is not, and should not be taken as an acknowledgement or any form of suggestion that prior art forms part of the common general knowledge of the person skilled in the art.

The entire content of all publications, patents, patent applications and other material recited in this specification is incorporated herein by reference.

### BRIEF DESCRIPTION OF THE SEQUENCE LISTING

SEQ ID NO: 1 is an amino acid consensus sequence for the mature domain of a Class II plant defensin.

SEQ ID NO: 2 is an exemplary full length amino acid sequence for the plant defensin NaD1, with

SEQ ID NO: 3 being the corresponding nucleic acid sequence.

SEQ ID NO: 4 is an exemplary amino acid sequence for the mature domain of the plant defensin NaD1, with SEQ ID NO: 5 being the corresponding nucleic acid sequence.

SEQ ID NO: 6 is an exemplary amino acid sequence for a recombinantly altered mature domain of the plant defensin NaD1, having an additional alanine residue at the N-terminal, with SEQ ID NO: 7 being the corresponding nucleic acid sequence.

SEQ ID NO: 8 is an exemplary full length amino acid sequence for the plant defensin TPP3, with SEQ ID NO: 9 being the corresponding nucleic acid sequence.

SEQ ID NO: 10 is an exemplary amino acid sequence for the mature domain of the plant defensin TPP3, with SEQ ID NO: 11 being the corresponding nucleic acid sequence.

SEQ ID NO: 12 is an exemplary amino acid sequence for a recombinantly altered mature domain of the plant defensin TPP3, having an additional alanine residue at the N-terminal, with SEQ ID NO: 13 being the corresponding nucleic acid sequence.

SEQ ID NO: 14 is an exemplary full length amino acid sequence for the plant defensin PhD1A, corresponding to Sol Genomics Network database accession number SGN-U207537, with SEQ ID NO: 15 being the corresponding nucleic acid sequence.

SEQ ID NO: 16 is a further exemplary full length amino acid for the plant defensin PhD1A that was cloned and sequenced by the inventors, with SEQ ID NO: 17 being the corresponding nucleic acid sequence.

SEQ ID NO: 18 is an exemplary amino acid sequence for the mature domain of the plant defensin PhD1A, with SEQ ID NO: 19 being the corresponding nucleic acid sequence.

SEQ ID NO: 20 is an exemplary full length amino acid sequence for the plant defensin NsD1, with SEQ ID NO: 21 being the corresponding nucleic acid sequence.

SEQ ID NO: 22 is an exemplary amino acid sequence for the mature domain of the plant defensin NsD1, with SEQ ID NO: 23 being the corresponding nucleic acid sequence.

SEQ ID NO: 24 is an exemplary full length amino acid sequence for the plant defensin NsD2, with SEQ ID NO: 25 being the corresponding nucleic acid sequence.

SEQ ID NO: 26 is an exemplary amino acid sequence for the mature domain of the plant defensin NsD2, with SEQ ID NO: 27 being the corresponding nucleic acid sequence.

### BRIEF DESCRIPTION OF THE FIGURES

The present invention will now be described, by way of example only, with reference to the following figures.

FIG. 1A is an immunoblot depicting expression and purification of recombinant NaD1 (rNaD1). P. pastoris expression medium collected at 48 h (30 μL) as well as samples from various stages of SP sepharose purification including the unbound fraction (30 μL), wash fraction (30 μL) and the first five 1.5 mL elution fractions (30 μL of each) were separated by SDS-PAGE and examined by immunoblotting with the α-NaD1 antibody. NaD1 from flowers (200 ng) was used as a positive control. Recombinant NaD1 could be detected in the 48 hour expression media as well as the SP sepharose elution fractions. FIG. 1B: is a reverse phase HPLC trace illustrating purity of rNaD1 purified from P. pastoris using SP sepharose. SP Sepharose elution fractions containing rNaD1 were loaded onto an analytical C8 RP-HPLC column and eluted using a 40 min linear gradient (0-100% buffer B). Proteins were detected by absorbance at 215 nm. A single major protein was detected indicating the protein was highly pure. FIG. 1C: FIG. 1C compares the structure of rNaD1 to native NaD1 purified from flowers. The far UV circular dichroism spectra of rNaD1 (open squares) and native NaD1 (closed diamonds) was compared and demonstrated no significant differences indicating that rNaD1 was correctly folded. FIG. 1D: FIG. 1D compares the antifungal activity of rNaD1 to native NaD1 purified from flowers. Hyphal growth of Fusarium oxysporum f.sp. vasinfectum in the presence of rNaD1 (open squares) or NaD1 (closed diamonds) is plotted relative to the growth of a no protein control for the same period. Graph represents data from three separate experiments performed in quadruplicate. Error bars represent standard error of the mean.

FIGS. 2A through 2E are graphical representations showing the effect of NaD1 on tumour cell viability. (2A) human breast carcinoma MCF-7, (2B) human colon carcinoma HCT-116, (2C) human melanoma MM170, (2D) human prostate carcinoma PC3, (2E) mouse melanoma B16-F1. MTT cell viability assays were performed on tumour cells that have been cultured in the presence of increasing concentrations (0 to 100 μM) of NaD1, rNaD1, or recombinant StPin1A (rStPin1A). % viability is shown having designated untreated cells as 100% viable. FIG. 2F provides a comparison of NaD1 activity against tumour cells and normal cells. Inhibitory concentrations (IC₅₀) (AM) of NaD1 or rNaD1 were determined from MTT cell viability assays on a range of human and mouse tumour cell lines and human normal primary cell lines. FIG. 2G is a graphical representation showing the effect of NaD1 and NaD2 against the human melanoma MM170. MTT cell viability assays were performed on cells cultured in the presence of increasing concentrations (0 to 100 μM) of NaD1, rNaD1 or NaD2. % viability is shown having designated untreated cells as 100% viable. FIGS. 2H and 2I show the effect of NaD1 on normal primary human cells (2H) umbilical vein endothelial cells (HUVEC), (21) coronary artery smooth muscle cells (CASMC). MTT cell viability assays were performed on cells cultured in the presence of increasing concentrations (0 to 100 μM) of NaD1, rNaD1, or rStPin1A. % viability is shown having designated untreated cells as 100% viable. FIG. 2J shows the effect of reduced and alkylated NaD1 (NaD1_{R&A}) on mouse melanoma B16-F1 cell viability. MTT cell viability assays were performed on cells that have been cultured in the presence of increasing concentrations (0 to 30 μM or 0 to 50 μM) of NaD1, or NaD1_{R&A} or rNaD1, respectively. % viability is shown having designated untreated cells as 100% viable.

FIGS. 3A and 3B are graphical representations showing the effect of NaD1 on the permeabilisation of (3A) human U937 myelomonocytic cells, or (3B) human melanoma cancer MM170 cells. Cells were incubated with increasing concentrations of NaD1 (0 to 100 μM) for 30 min at 37° C. upon which propidium iodide (PI) was added. The number of cells that stained positively for PI (PI⁺) were determined by flow cytometry. FIGS. 3C and 3D show the effect of (3C) NaD1 and (3D) NaD1_{R&A} on the release of ATP from U937 human myelomonocytic cells. NaD1 or NaD1_{R&A} were added to cells in phosphate buffered saline (PBS) together with an ATP luciferase detection reagent (Roche™) and the release of ATP detected by over time by spectrophotometry at a wavelength of 562 nm. FIG. 3E Field-emission scanning electron microscopy was used for the imaging of morphological changes in PC3 cells treated with NaD1. Left and right panels are FE-SEM images of untreated or NaD1-treated PC3 cells, respectively. Top panels are of cells at 1,200× magnification and the low secondary electron image (LEI) of the microscope was 10 um at an accelerating voltage of 2.00 kV. The bottom panels are of cells at 3000× magnification and the low secondary electron image (LEI) of the microscope was 1 um at an accelerating voltage of 2.00 kV.

FIG. 4 is a graphical representation showing the effect of NaD1 and rNaD1 on red blood cell (RBC) lysis. Human RBCs were incubated with increasing concentrations of NaD1, rNaD1, PBS alone, or water, for 16 h at 37° C. Released haemoglobin indicative of RBC lysis was then determined by spectrophotometry at a wavelength of 412 nm. Results have been normalised to RBCs treated with water (designated 100% lysis).

FIG. 5 is a graphical representation showing the effect of NaD1 on the permeabilisation of tumour cells in the presence of serum. U937 cells in the presence of 10 μM NaD1 were incubated with increasing concentrations of foetal calf serum (FCS) for 30 min at 37° C. upon which propidium iodide (PI) was added. The number of cells that stained positively (PI⁺) or negatively (PI⁻) were determined by flow cytometry.

FIG. 6 is a graphical representation of the effect of NaD1 on B16-F1 tumour growth. Solid B16-F1 melanoma tumours (˜10 mm in diameter) were established subcutaneously in C57BL/6 mice. Tumours were then injected intratumourally with 50 μL of PBS containing 1 mg/mL of NaD1, NaD1_{R&A}, or just PBS vehicle alone every 2 days and the effect on tumour growth determined by measurement of tumour size. Tumour size was normalised to 1 for each mouse at day 0. Results represent standard error of the mean on five mice per treatment.

FIGS. 7A through 7C are graphical representations showing the binding of NaD1 to cellular lipids. Echelon™ lipid strips were probed with NaD1 and binding was detected with a rabbit anti-NaD1 antibody followed by a horseradish peroxidise (HRP) conjugated donkey anti-rabbit IgG antibody. (7A) Membrane lipid Strip™, (7B) PIP lipid Strip™, (7C) SphingoStrip lipid Strip™. Binding of NaD1 to individual lipids on each strip was quantitated by densitometry. FIGS. 7D through 7F show the binding of NaD2 to cellular lipids. Echelon™ lipid strips were probed with NaD2 and binding was detected with a rabbit anti-NaD2 antibody followed by a HRP conjugated donkey anti-rabbit IgG antibody. (7D) Membrane lipid Strip™, (7E) PIP lipid Strip™, (7F) SphingoStrip lipid Strip™. Binding of NaD2 to individual lipids on each strip was quantitated by densitometry. FIG. 7G summarises the relative lipid binding specificity and strength of native, recombinant, and reduced and alkylated NaD1 and NaD2, native NsD3, NsD1, NsD2, PhD1A and TPP3. Bars indicate strength of binding. PS, phosphatidylserine; PA, phosphatidylalanine; PG, phosphatidylglycerol.

FIG. 8 is a diagrammatic representation of the structure of the precursor proteins of the two major classes of plant defensins, as predicted from cDNA clones. In the first and largest class, the precursor protein is composed of an endoplasmic reticulum (ER) signal sequence and a mature defensin domain. (8A). The second class of defensins are produced as larger precursors with C-terminal propeptides (CTPPs) (8B).

FIG. 9A is a graphical representation showing the effect of PhD1A on the permeabilisation of human U937 myelomonocytic cells. Cells were incubated with increasing concentrations of native PhD1A (0 to 50 μM) for 30 min at 37° C. upon which propidium iodide (PI) was added. The number of cells that stained positively for PI (PI⁺) was determined by flow cytometry. FIG. 9B is a graphical representation showing the effect of PhD1A on the release of ATP from U937 human myelomonocytic cells. PhD1A was added to cells in PBS together with an ATP luciferase detection reagent (Roche™) and the release of ATP detected over time by spectrophotometry at a wavelength of 562 nm. FIG. 9C is a graphical representation showing the effect of recombinant (rTPP3) on the permeabilisation of human U937 myelomonocytic cells. Cells were incubated with increasing concentrations of rTPP3 (0 to 40 μM) for 30 min at 37° C. upon which propidium iodide (PI) was added. The number of cells that stained positively for PI (PI⁺) was determined by flow cytometry. FIG. 9D is a graphical representation showing the effect of rTPP3 on the release of ATP from U937 human myelomonocytic cells. Recombinant TPP3 was added to cells in PBS together with an ATP luciferase detection reagent (Roche™) and the release of ATP detected by over time by spectrophotometry at a wavelength of 562 nm.

FIG. 10 is a graphical representation showing the effect of solanaceous Class II defensins (NaD1, PhD1A, TPP3), and non-solanaceous Class I defensins Dahlia merckii defensin Dm-AMP1, Hordeum vulgare gamma-thionin γ1-H, Zea mays gamma-thionin γ2-Z on the permeabilisation of human U937 myelomonocytic cells. Cells were incubated with 10 μM each molecule for 30 min at 37° C. upon which propidium iodide (PI) was added. The number of cells that stained positively for PI (PI⁺) was determined by flow cytometry. Data is the mean of three replicates±SEM.

FIGS. 11A and 11B are graphical representations showing the effect of PhD1A (11A) or rTPP3 (11B) on the permeabilisation of tumour cells in the presence of serum. U937 cells in the presence or absence of 10 μM PhD1A or rTPP3 were incubated with increasing concentrations of foetal calf serum (FCS) for 30 min at 37° C. upon which propidium iodide (PI) was added. The number of cells that stained positively (PI⁺) or negatively (PI⁻) was determined by flow cytometry. The high number of permeabilised cells without defensin at 0% FCS is a result of the absence of serum.

FIG. 12A is a graphical representation showing the effect of native NsD3, NsD1, NsD2 compared to native NaD1 on the release of ATP from U937 human myelomonocytic cells. Each defensin was added to cells at 10 μM in PBS together with an ATP luciferase detection reagent (Roche™) and the release of ATP detected over time by spectrophotometry at a wavelength of 562 nm. FIG. 12B is a graphical representation showing the effect of NsD3, NsD1, NsD2 compared to NaD1 on the permeabilisation of human U937 myelomonocytic cells. Cells were incubated with 100 for 30 min at 37° C. upon which propidium iodide (PI) was added. The number of cells that stained positively for PI (PI+) was determined by flow cytometry.

FIG. 13 is a graphical representation showing the effect of the class II defensins NsD1, NsD2, PhD1A and NaD1 on red blood cell (RBC) lysis. Human RBCs were incubated with 10 μM or 30 μM of each defensin for 16 h at 37° C. Released haemoglobin indicative of RBC lysis was then determined by spectrophotometry at a wavelength of 412 nm. Results have been normalised to RBCs treated with water (designated 100% lysis). PBS=negative (or background lysis) control.

FIGS. 14A through 14E are graphical representations showing the binding of NsD1 (a), NsD2 (b), NsD3 (c), TPP3 (d) and PhD1a (e) to PIP cellular lipids. PIP Echelon™ lipid strips were probed with defensins and binding was detected with a rabbit anti-NaD1 antibody (for NsD1, NsD2, PhD1A, TPP3) or rabbit anti-NaD2 antibody (for NsD3) followed by a horseradish peroxidise (HRP) conjugated donkey anti-rabbit IgG antibody. Binding of defensins to individual lipids on each strip was quantitated by densitometry.

FIG. 15 is an amino acid sequence alignment of the mature domains of Class I and Class II plant defensins. NaD1 and NaD2 (Nicotiana alata), NsD1, NsD2, NsD3 (Nicotiana suaveolens), PhD1A (Petunia hybrida), TPP3 (Solanum lycopersicum), Dm-AMP1 (Dahlia merckii). Identity or homology is indicated by black- or grey-boxed residues, respectively. Conserved disulfide bonds are shown as solid lines.

### DETAILED DESCRIPTION OF THE INVENTION

The inventors have surprisingly found that defensins, also known as γ-thionins, have potent cytotoxic properties. These significant findings describe a novel and important way in which proliferative diseases may be prevented and treated. Accordingly, these findings provide for methods for the prevention or treatment of proliferative diseases such as cancer, as well as associated uses, systems and kits.

For example, NaD1 is a plant defensin isolated from floral tissue of Nicotiana alata. The amino acid and coding sequences of NaD1 are disclosed in International Patent Publication No. WO 02/063011, the entire contents of which are incorporated by reference herein.

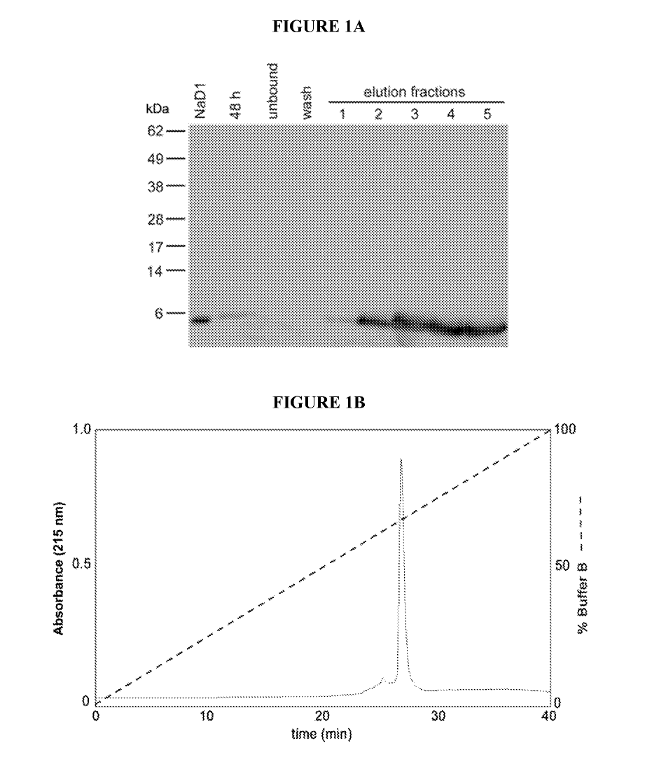

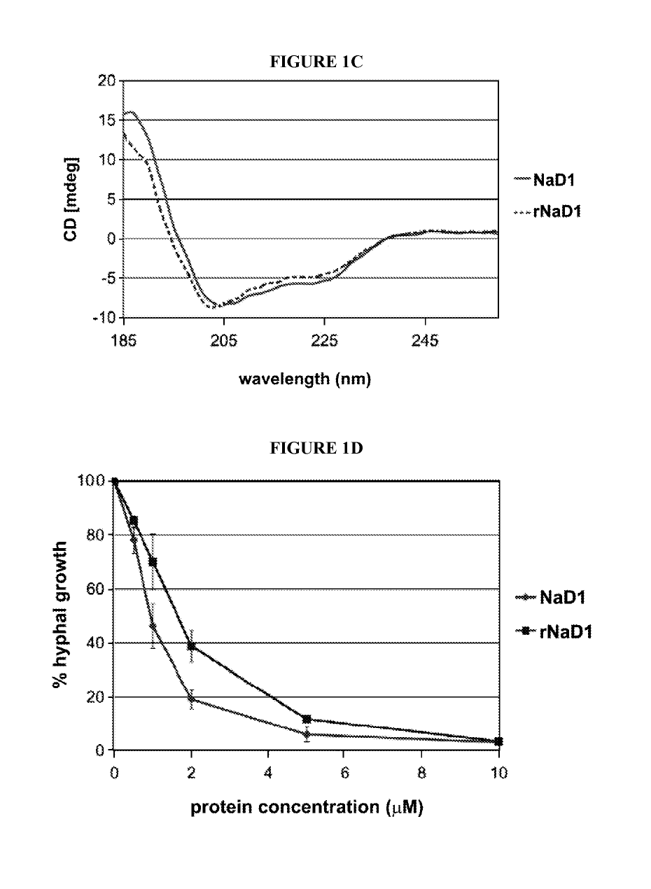

The ability to produce large quantities of active defensins such as NaD1 is of fundamental importance when considering potential use as a therapeutic in a clinical setting. The purification of the required large amounts of NaD1 from its natural source (flowers of the ornamental tobacco N. alata) is not feasible, necessitating the production of active recombinant protein. A Pichia pastoris expression system combined with a defined protein purification approach has been successfully established to produce high levels of pure active recombinant NaD1 (FIGS. 1A, B). The recombinant NaD1 has a similar structural fold to that of native NaD1 (FIG. 1C) and retains its ability to inhibit hyphal growth of F. oxysporum (FIG. 1D). These data demonstrate the establishment of an efficient system for the production of large amounts of pure active recombinant defensins such as NaD1.

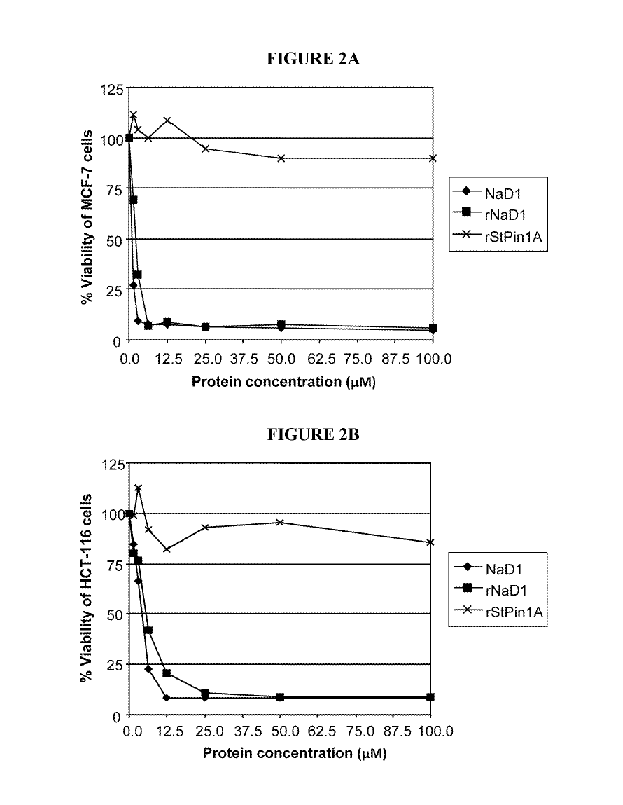

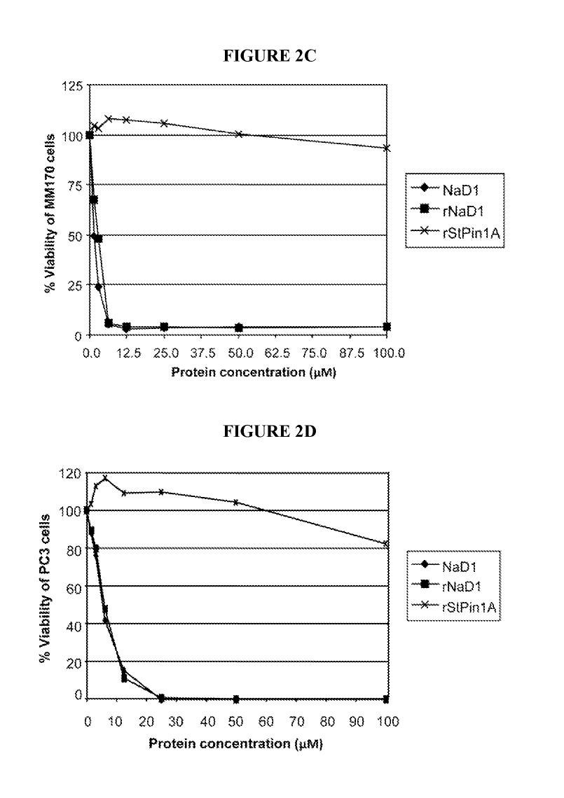

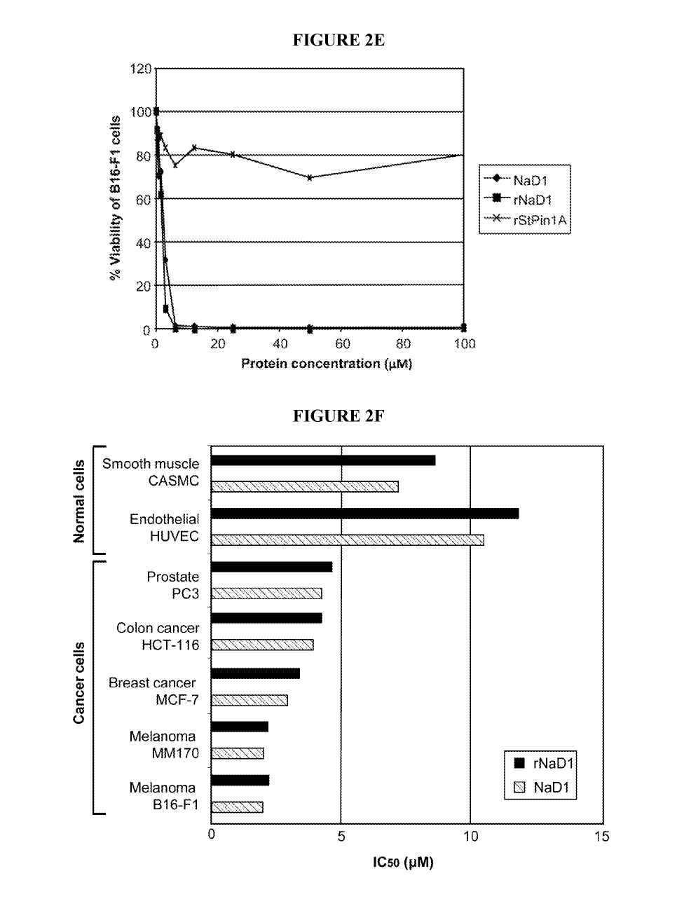

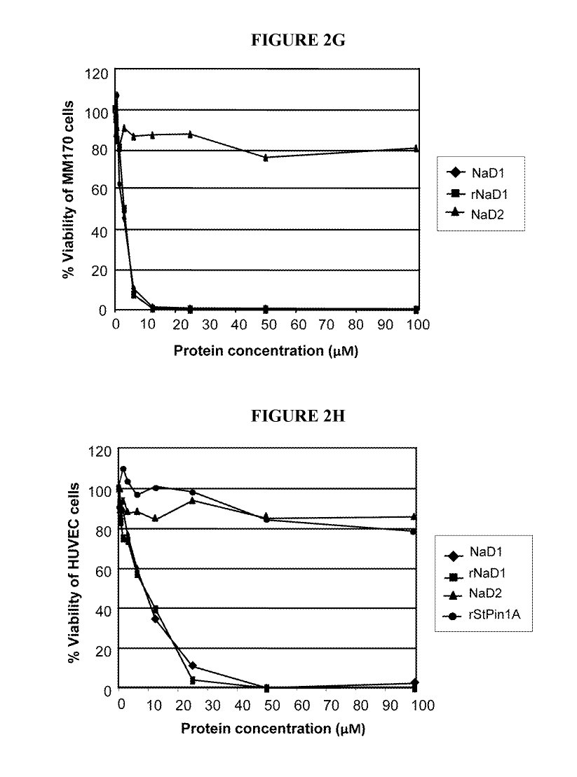

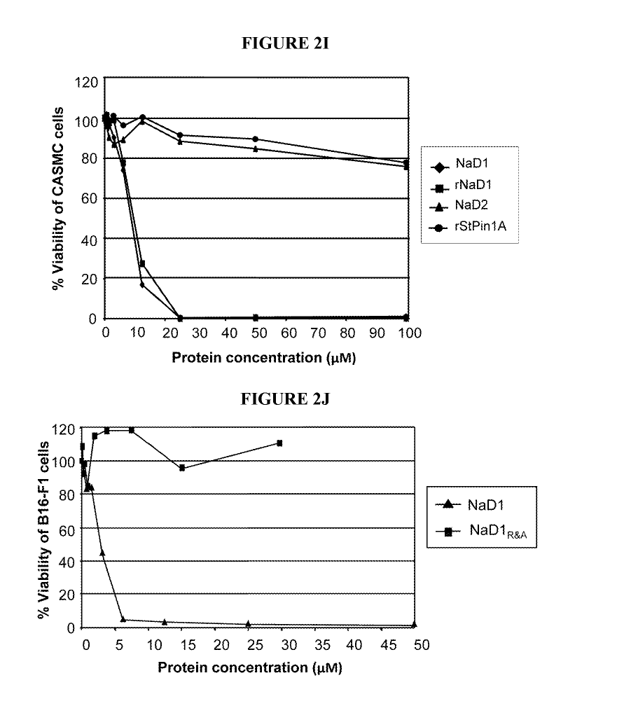

Native and recombinant NaD1 were shown to selectively kill tumour cells in vitro at low μM concentrations (FIGS. 2A-F). A range of human tumour cell lines of different tissue origin (prostate carcinoma PC3, colon carcinoma HCT-116, breast carcinoma MCF-7, and melanoma MM170) and the mouse melanoma cell line B16-F1 were all killed at similar efficiencies by both native or recombinant NaD1 at IC₅₀ values of between 2 and 4.5 μM. Normal primary cells (human coronary artery smooth muscle or umbilical vein endothelial cells) were also killed by native or recombinant NaD1 but required significantly higher concentrations (IC₅₀ values of 7.5-12 μM) than for tumour cell lines. These data indicate that plant defensins such as NaD1 exhibit potential as anti-cancer agents that, when used at a specific low μM concentration, could be applied to selectively kill tumour cells but not normal cells. In contrast to NaD1 (a solanaceous Class II defensin) the solanaceous Class I defensin NaD2 or the protease inhibitor StPin1A showed no ability to kill tumour cells (FIGS. 2A-I), suggesting that Class II defensins have a unique capacity to kill tumour cells (discussed further below). A reduced and alkylated form of NaD1 did not affect tumour cell viability, demonstrating that an intact tertiary structure is critical for the tumour cell cytotoxicity of NaD1.

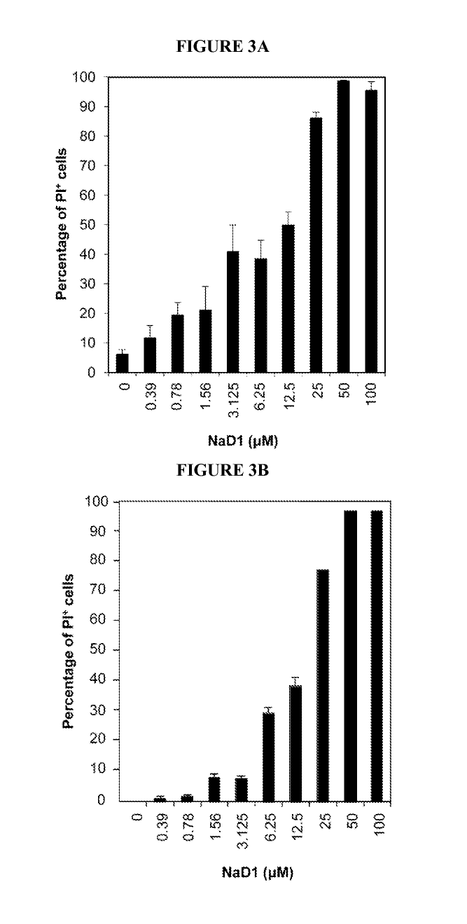

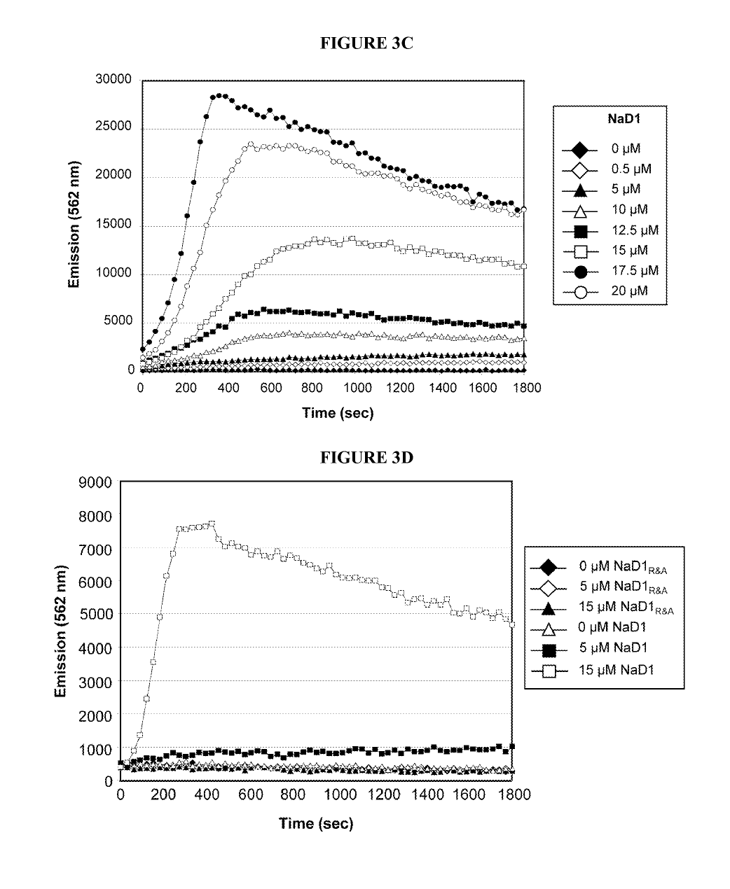

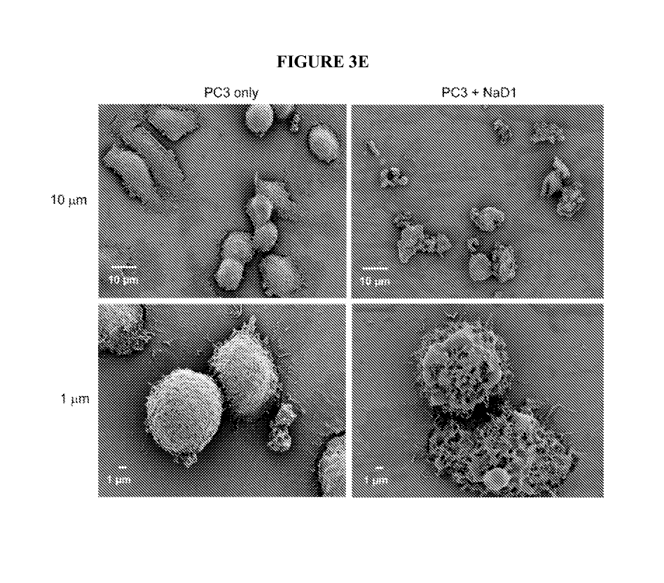

The mechanism of action of NaD1 on tumour cells was investigated and found to involve permeabilisation of the plasma membrane. NaD1 permeabilised the human tumour cell lines U937 and MM170 in a dose-dependent manner as demonstrated by the ability of NaD1 to mediate both the uptake of the fluorescent dye PI (FIGS. 3A, 3B) and the release of ATP (FIGS. 3C, 3D). The permeabilisation of tumour cells was rapid, with ATP being released immediately upon addition to cells with the peak of ATP release at ˜5 min. A reduced and alkylated form of NaD1 was not able to permeabilise tumour cells (FIG. 3D). Further support for the tumour cell permeabilisation activity of NaD1 was provided by the examination of human prostate carcinoma PC3 cells treated with NaD1 using scanning electron microscopy (FIG. 3E). These data show that NaD1 kills tumour cells by rapidly destabilising the plasma membrane leading to cell permeabilisation. The understanding of the mechanism of NaD1 action provides valuable information for therapeutic uses of defensins in isolation or in combination with other anti-cancer drugs.

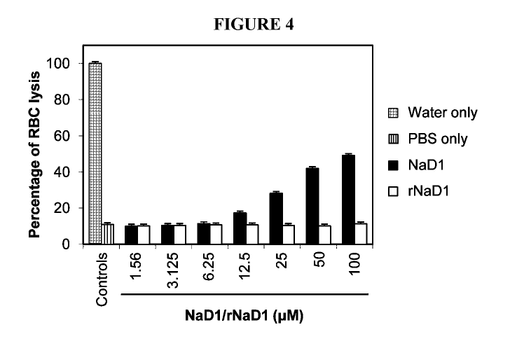

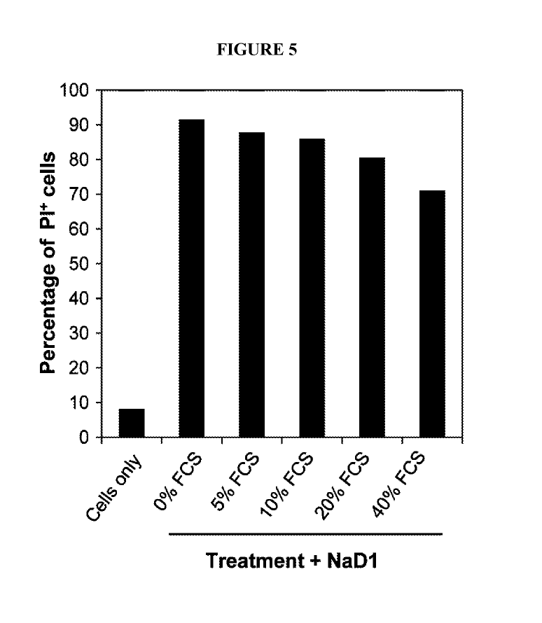

The potential for the application of defensins such as NaD1 as anti-cancer agents also necessitates that they retain activity in serum/plasma and do not show lytic activity on red blood cells. NaD1 showed no haemolytic activity against human red blood cells (RBC) at the concentrations required to kill tumour cells in vitro. At concentrations of 12.5 μM and above, native NaD1 showed haemolytic activity, peaking at ˜50% RBC lysis at 100 μM. Significantly, recombinant NaD1 showed no haemolytic activity even at high concentrations up to 100 μM (FIG. 4). Native and recombinant NaD1 differ in primary amino acid sequence by the addition of a single alanine residue to the N-terminus of recombinant NaD1. As there appears to be no major structural difference between native and recombinant NaD1 (FIG. 1C) and both forms show very similar activity in permeabilising tumour cells, the additional alanine at the N-terminus of recombinant NaD1 may be responsible for the loss of the haemolytic activity of NaD1. As such, the production of recombinant defensins such as NaD1 with an alanine on N-termini is predicted to have a significant advantage over native defensin sequences in terms of application as a therapeutic with minimal haemolytic activity. It should also be noted that both native and recombinant NaD1 retained the ability to kill tumour cells in the presence of up to 40% serum (FIG. 5). The retention of the tumour cell permeabilisation activity of NaD1 in the presence of serum is an important observation, as many cationic peptides have been shown to have greatly reduced activity in the presence of serum and are rendered ineffective as therapeutic agents.

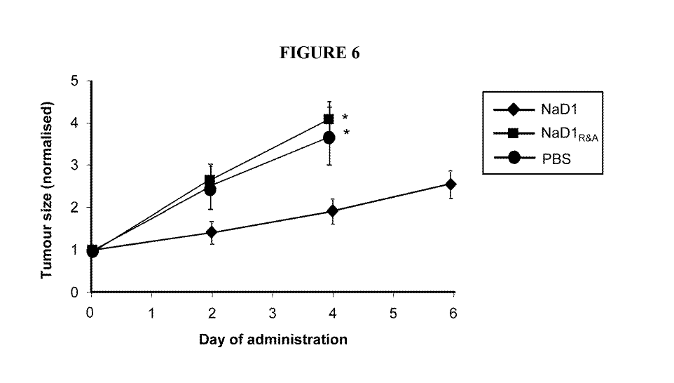

The potential for defensins such as NaD1 as anti-cancer agents was further demonstrated in an in vivo model of melanoma growth in mice. The treatment of solid advanced B16F1 tumours by the direct intra-tumour injection of 1 mg NaD1/kg body weight resulted in a significant reduction in tumour growth when compared to tumours treated with reduced and alkylated NaD1 (inactive) or vehicle alone (FIG. 6). Furthermore, NaD1 was shown to have no adverse effects on mice when administered orally at up to 300 mg NaD1/kg body weight.

The data shown herein demonstrate (i) broad in vitro tumour cell selectivity at low μM concentration, (ii) retention of activity in the presence of serum, and (iii) lack of haemolytic activity, and therefore make defensins such as NaD1 promising models as anti-cancer agents.

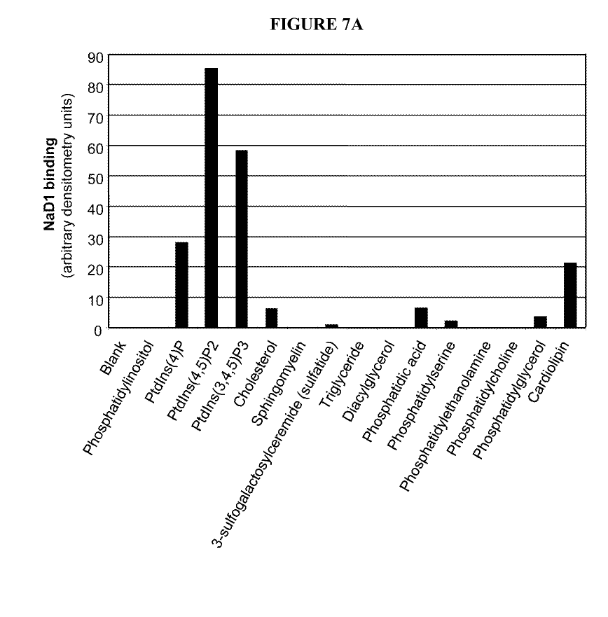

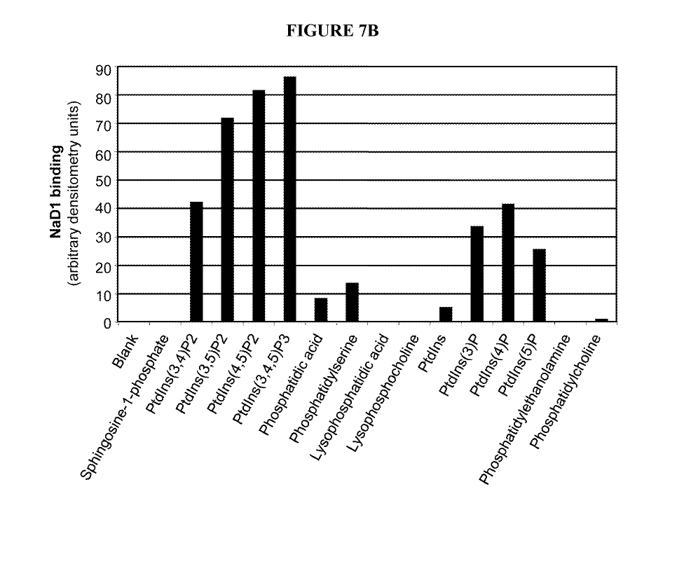

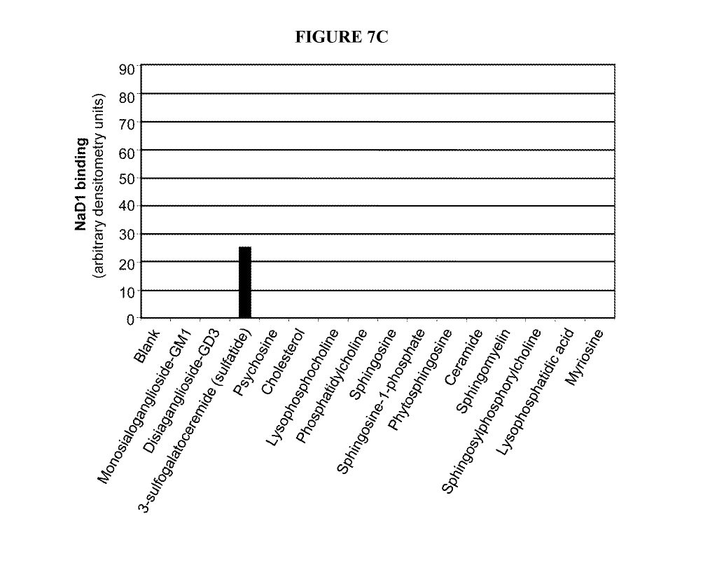

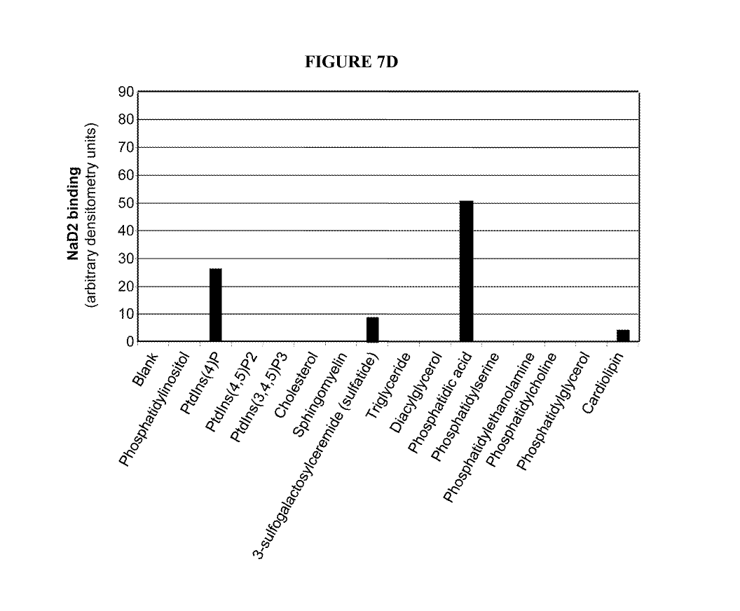

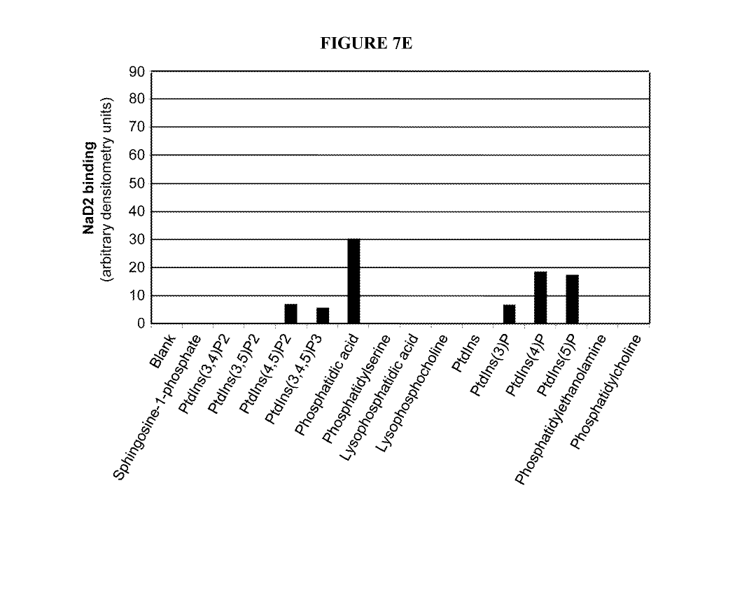

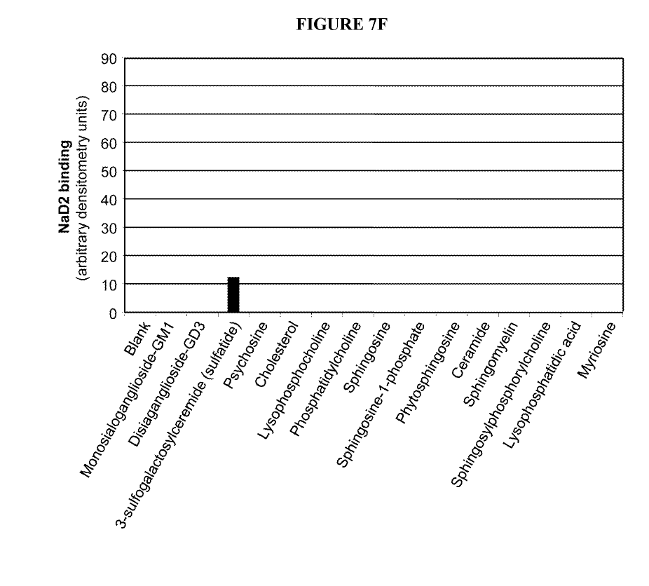

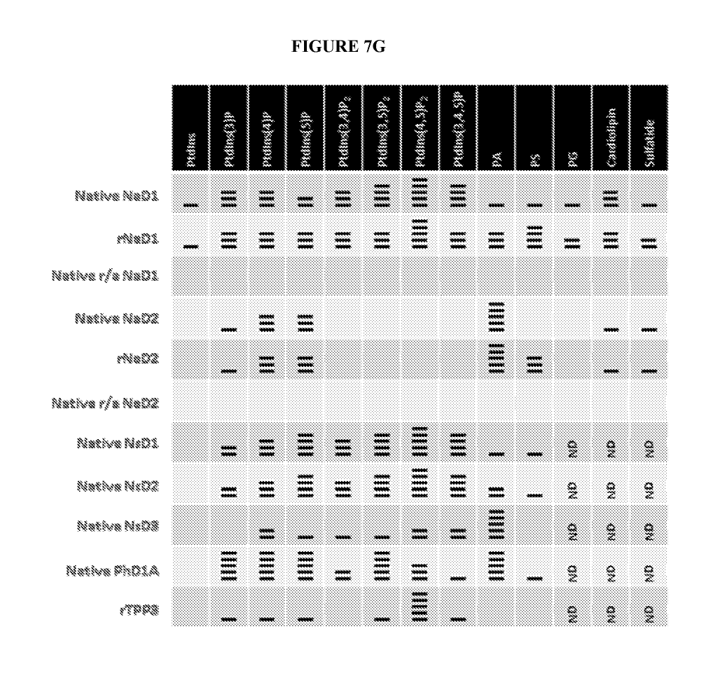

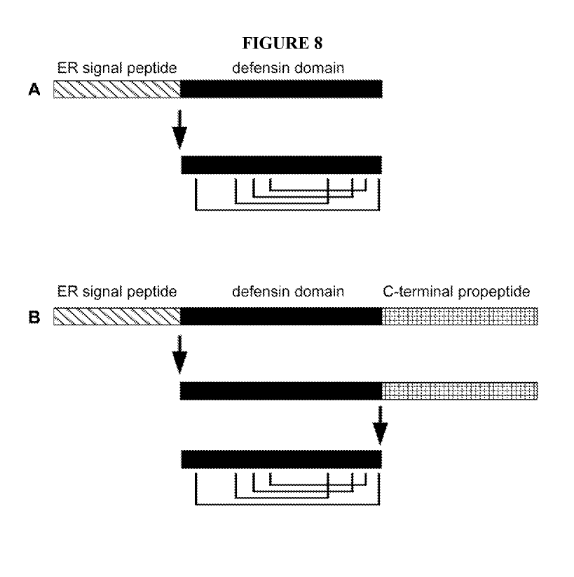

The investigation of candidate NaD1-interacting molecules led to the identification of phospholipids as ligands of NaD1. NaD1 was found to bind specifically to a range of phosphophoinositides as well as phosphatidylserine (PS), phosphatidyl alanine (PA), phosphatidylglycerol (PG) and sulfatide (FIGS. 7A-C). Both the native and recombinant NaD1 showed very similar lipid binding specificity (FIG. 7G). Interestingly, the class I defensin NaD2 was also found to bind phospholipids but with a very distinct specificity to NaD1, with strong binding observed to PA but not to many of the phosphoinositides shown to bind NaD1 (FIGS. 7D-F). The interaction of NaD1 with this specific array of phospholipids may contribute to the tumour cell cytotoxic activity of NaD1. It should also be noted the reduction and alkylation of NaD1 resulted in loss of binding to phospholipids (FIG. 7G). These data suggest that the tertiary structure of NaD1 is essential for both phospholipid binding and anti-tumour activity.

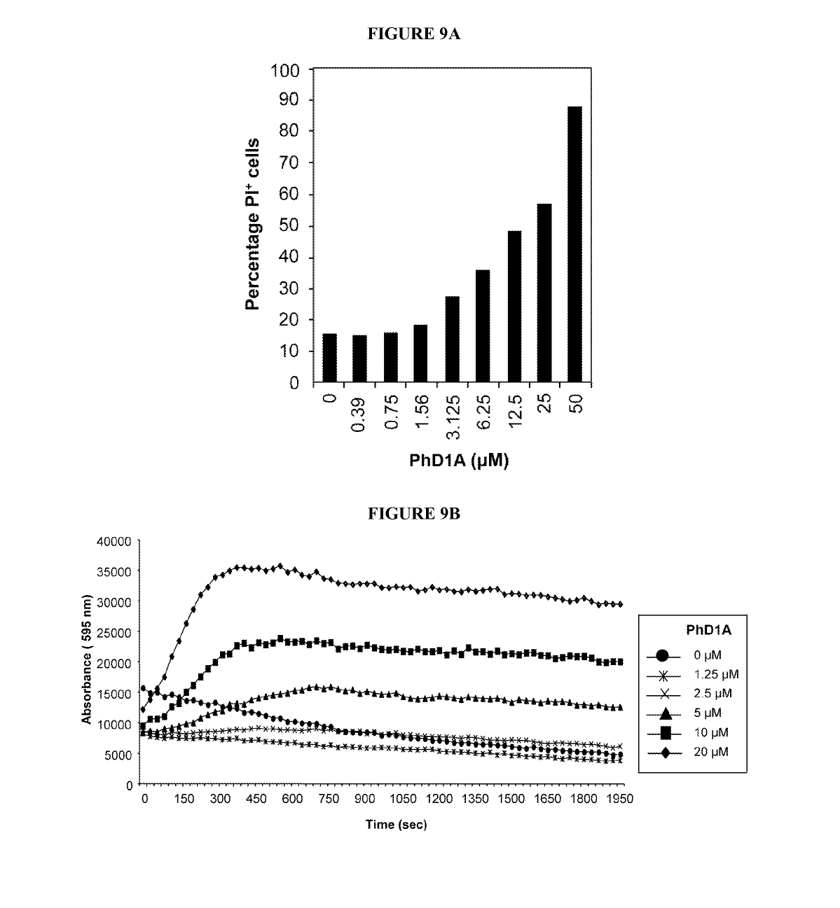

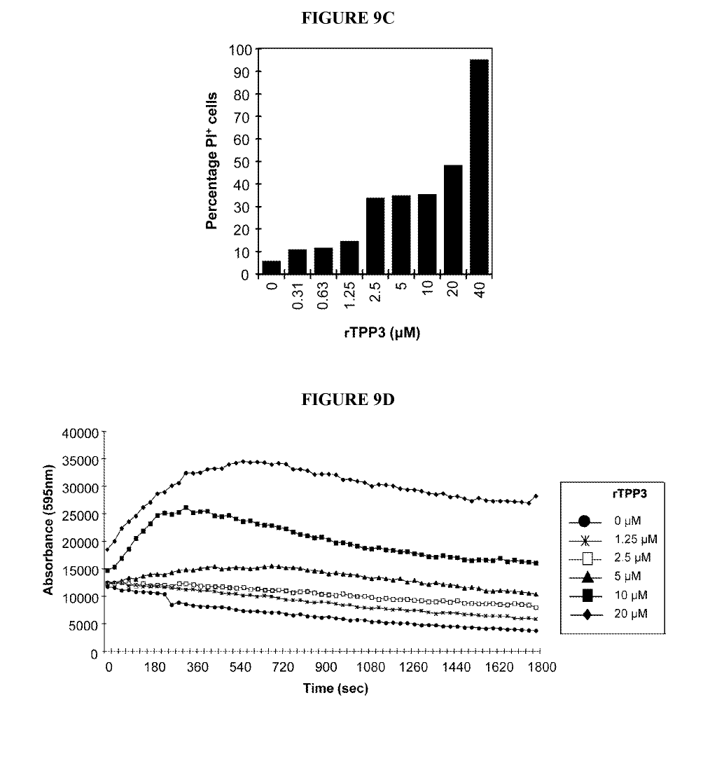

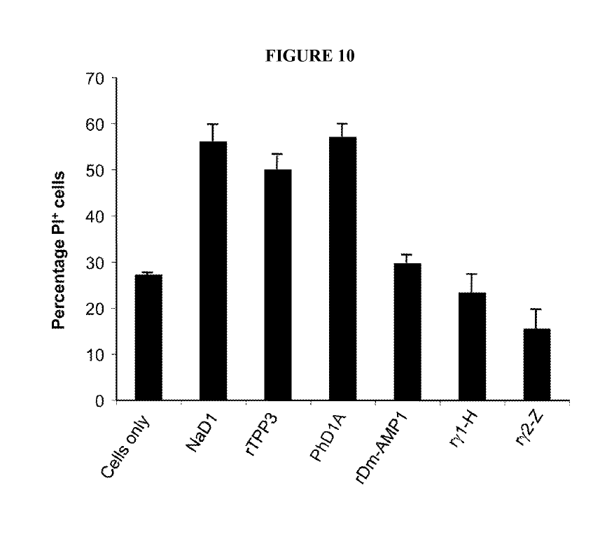

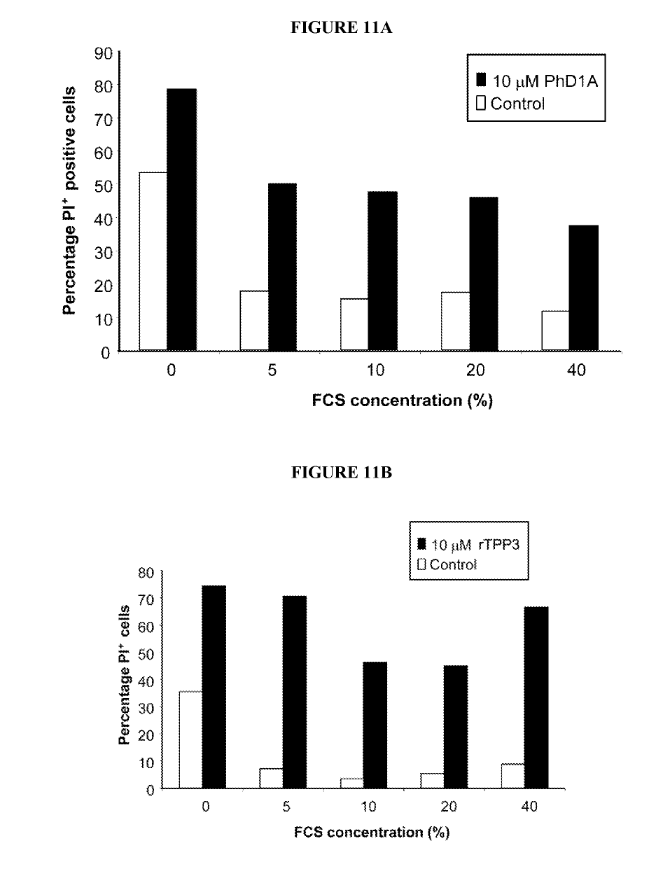

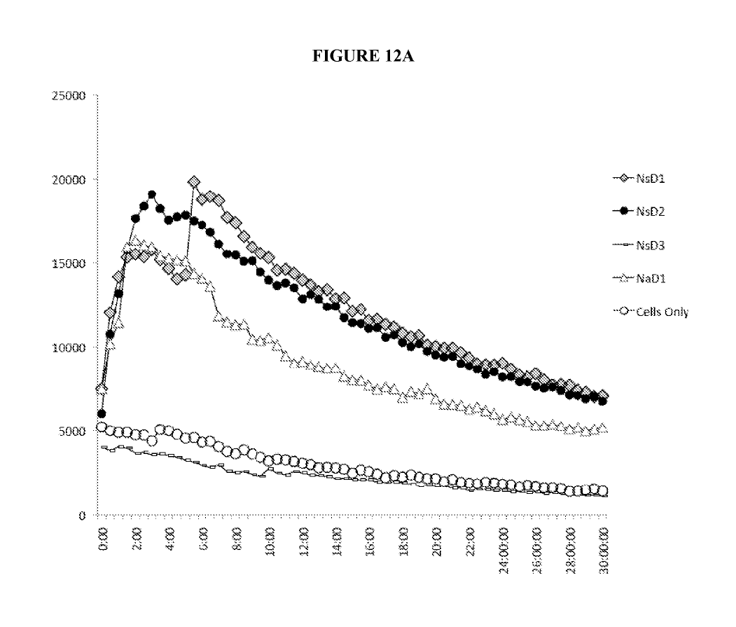

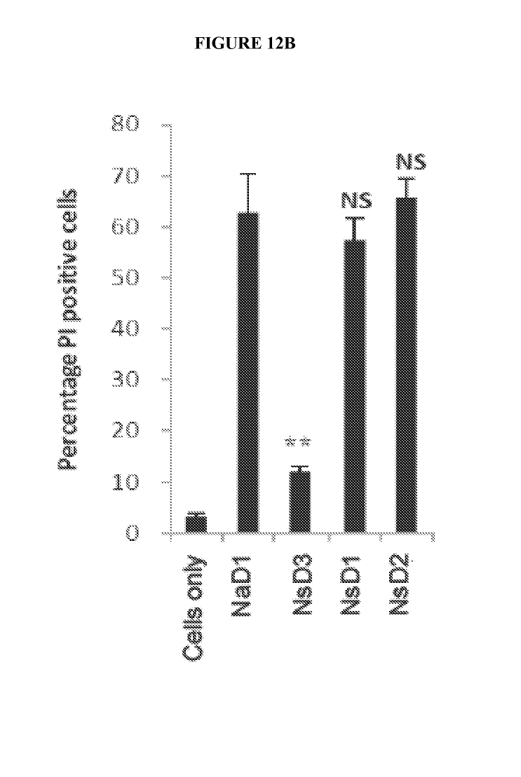

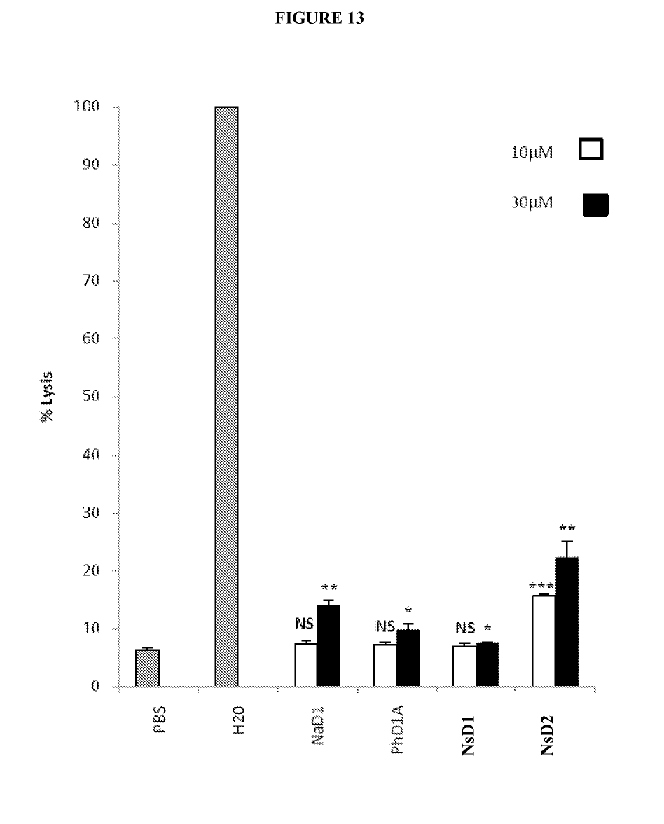

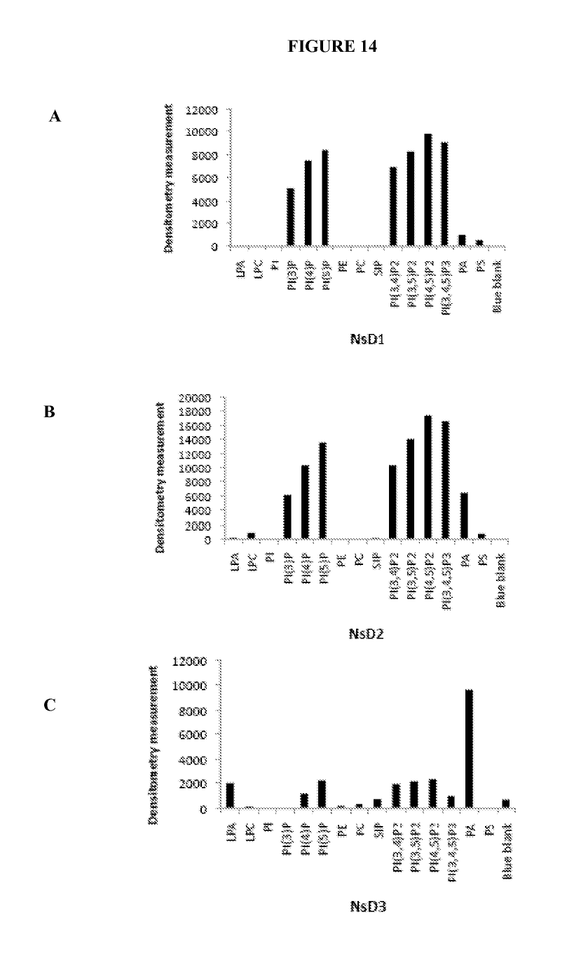

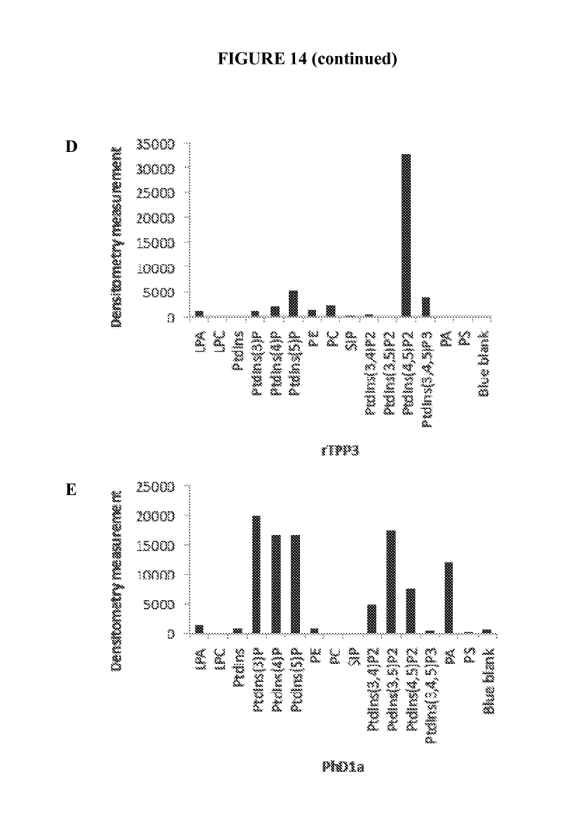

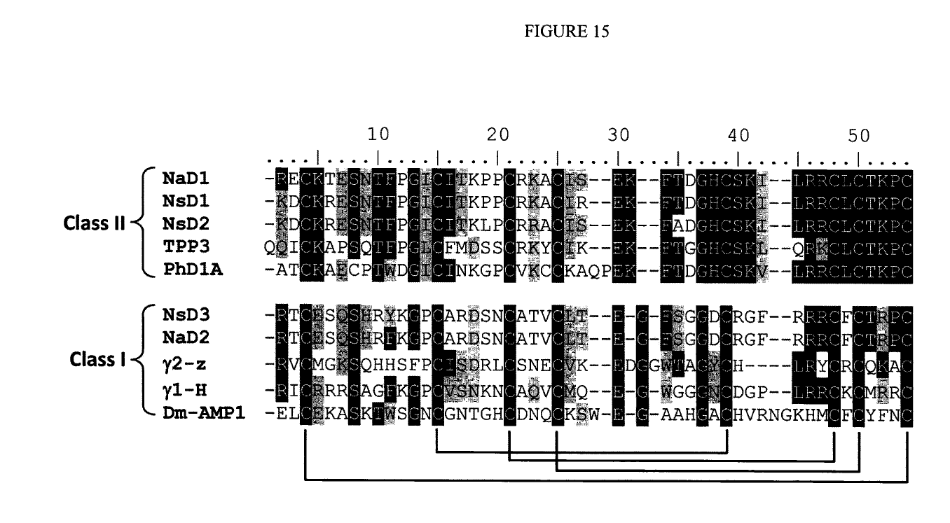

The ability of the solanaceous Class II defensin NaD1 to kill tumour cells but not the Class I defensin NaD2 suggested that the solanaceous Class II defensins may have particular cytotoxic activity towards tumour cells. Indeed, the solanaceous Class II defensins TPP3 and PhD1A were both found to have similar tumour cell permeabilisation activity as NaD1 (FIGS. 9A-D). As described for NaD1, both TPP3 and PhD1A were also found to retain tumour cell permeabilisation activity in the presence of serum (FIGS. 11A and B). In contrast, the non-Solanaceous Class I defensins Dm-AMP1, γ1-H and γ2-Z, showed no tumour cell permeabilisation activity (FIG. 10). Further supporting evidence that the ability to kill tumour cells is unique to the solanaceaous class II defensins and not class I defensins is demonstrated in that the class II solanaceaous defensins NsD1 and NsD2 permeabilised tumour cells but the class I defensin NsD3 did not (FIG. 12). It should also be noted that the observed lack of haemolytic activity of NaD1 on human red blood cells was conserved in other class II defensins. NsD1, NsD2 and PhD1A all showed no or very low ability to lyse RBCs up to concentrations of 30 μM (FIG. 13). In addition, the distinct pattern of phospholipid binding specificity identified for the class II defensin NaD1 and the class I defensin NaD2 (FIG. 7) was also observed for other solanaceaous class I and II defensins. The class II defensins NsD1, NsD2, Tpp3 and PhD1A all showed a general preference of binding to phosphoinositides (FIG. 14A-B, D-E) whereas the class I defensin NsD3 bound most strongly to PA (FIG. 14C).

### Plant Defensins for Use in Preventing or Treating a Proliferative Disease

The present invention provides plant defensins for use in preventing or treating a proliferative disease.

In some embodiments, the plant defensin is any plant gamma-thionin.

In other embodiments, the plant defensin has at least eight canonical cysteine residues which form disulfide bonds in the configuration: Cys_{I}-Cys_{VIII}, Cys_{II}-Cys_{IV}, Cys_{III}-Cys_{VI} and Cys_{V}-Cys_{VII}.

In yet other embodiments, the plant defensin is a Class II plant defensin with or having previously had a C-terminal prodomain or propeptide (CTPP).

In particular embodiments, the plant defensin is derived or derivable from Solanaceae, Poaceae or Asteraceae.

In some embodiments, the plant defensin is not CcD1 (NCBI database accession no AF128239).

In preferred embodiments, the plant defensin has at least eight canonical cysteine residues which form disulfide bonds in the configuration: Cys_{I}-Cys_{VIII}, Cys_{II}-Cys_{IV}, Cys_{III}-Cys_{VI} and Cys_{V}-Cys_{VII}, and is a Class II Solanaceous plant defensin with or previously having had a C-terminal prodomain or propeptide (CTPPs).

In some embodiments, the plant defensin comprises the amino acid sequence set forth as SEQ ID NOs:1, 2, 4, 6, 8, 10, 12, 14, 16, 18, 20, 22, 24 or 26 or a fragment thereof.

In yet other embodiments, the plant defensin comprises an amino acid sequence that is 95%, 90%, 85%, 80%, 75%, 70%, 65% or 60% identical to the amino acid sequence set forth as SEQ ID NOs: 1, 2, 4, 6, 8, 10, 12, 14, 16, 18, 20, 22, 24 or 26 or a fragment thereof.

In still other embodiments, the plant defensin comprises an amino acid sequence that is 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% identical to the amino acid sequence set forth as SEQ ID NOs: 1, 2, 4, 6, 8, 10, 12, 14, 16, 18, 20, 22, 24 or 26 or a fragment thereof.

In yet further embodiments, the plant defensin is a Solanaceous Class II defensin.

In particular embodiments, the plant defensin is derived or derivable from Nicotiana alata, Nicotiana suaveolens, Petunia hybrida, Solanum lycopersicum, Nicotiana tabacum, Nicotiana attenuate, Nicotiana excelsior, Nicotiana paniculata, Solanum tuberosum, Capsicum chinense or Capsicum annuum.

In more particular embodiments, the plant defensin is derived or derivable from Nicotiana alata, Nicotiana suaveolens, Petunia hybrida or Solanum lycopersicum.

In some embodiments, the defensin is selected from the group comprising NaD1 (NCBI database accession no. A509566), NsD1 (SEQ ID NO: 20 or 22), NsD2 (SEQ ID NO: 24 or 26), PhD1A (Sol Genomics Network database accession no. SGN-U207537 or SEQ ID NO: 16), TPP3 (NCBI database accession no. SLU20591), FST (NCBI database accession no. Z11748), NatD1 (NCBI database accession no. AY456268), NeThio1 (NCBI database accession no. AB005265), NeThio2 (NCBI database accession no. AB005266), NpThio1 (NCBI database accession no. AB005250), CcD1 (NCBI database accession no. AF128239), PhD1 (NCBI database accession no. A507975), PhD2 (NCBI database accession no. AF507976), any defensin with an amino acid or nucleic acid sequence corresponding to any of the sequences set forth under NCBI database accession numbers EU367112, EU560901, AF112869 or AF112443, or any defensin with an amino acid or nucleic acid sequence corresponding to any of the sequences set forth under Sol Genomics Network database accession numbers SGN-U448338, SGN-U449253, SGN-U448480, SGN-U447308, SGN-U578020, SGN-U577258, SGN-U286650, SGN-U268098, SGN-U268098, SGN-U198967, SGN-U196048, SGN-U198968 or SGN-U198966.

In particularly preferred embodiments, the plant defensin is NaD1, NsD1, NsD2, PhD1A or TPP3.

In some embodiments, the plant defensin may be a fragment of any amino acid sequence or a fragment or complement of any nucleic acid sequence disclosed herein.

In particular embodiments, the fragment may comprise a mature domain.

In preferred embodiments, the amino acid sequence of the mature domain is set forth as SEQ ID NOs: 4, 6, 10, 12, 18, 22 or 26.

In some embodiments, the plant defensin may be an isolated, purified or recombinant plant defensin.

In particular embodiments, the recombinant plant defensin has an additional alanine residue at or near the N-terminal end.

In preferred embodiments, the recombinant plant defensin has reduced haemolytic activity.

In particularly preferred embodiments, the recombinant plant defensin comprises the amino acid sequence set forth as SEQ ID NO: 6, 22 or 26, or a fragment thereof.

### Polynucleotides

In embodiments where the compositions of the present invention comprise polypeptides, the present invention also provides nucleic acids encoding such polypeptides, or fragments or complements thereof. Such nucleic acids may be naturally occurring or may be synthetic or recombinant.

In some embodiments, the nucleic acids may be operably linked to one or more promoters. In particular embodiments, the nucleic acids may encode polypeptides that prevent or treat proliferative diseases.

In some embodiments, the plant defensin is therefore provided in the form of a nucleic acid. In some embodiments, the plant defensin nucleic acid encodes the amino acid sequence set forth as SEQ ID NOs: 1, 2, 4, 6, 8, 10, 12, 14, 16, 18, 20, 22, 24 or 26 or a fragment thereof. In yet other embodiments, the plant defensin nucleic acid comprises the nucleotide sequence set forth as SEQ ID NOs: 3, 5, 7, 9, 11, 13, 15, 17, 19, 21, 23, 25 or 27 or a fragment or complement thereof.

In yet other embodiments, the plant defensin nucleic acid comprises a nucleotide sequence that is 95%, 90%, 85%, 80%, 75%, 70%, 65% or 60% identical to the nucleotide sequence set forth as SEQ ID NOs: 3, 5, 7, 9, 11, 13, 15, 17, 19, 21, 23, 25 or 27 or a fragment or complement thereof.

In still other embodiments, the plant defensin nucleic acid comprises a nucleotide sequence that is 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% identical to the nucleotide sequence set forth as SEQ ID NOs: 3, 5, 7, 9, 11, 13, 15, 17, 19, 21, 23, 25 or 27 or a fragment or complement thereof.

### Vectors, Host Cells and Expression Products

The present invention also provides vectors comprising the nucleic acids as set forth herein. The vector may be a plasmid vector, a viral vector, or any other suitable vehicle adapted for the insertion of foreign sequences, its introduction into cells and the expression of the introduced sequences. The vector may be a eukaryotic expression vector and may include expression control and processing sequences such as a promoter, an enhancer, ribosome binding sites, polyadenylation signals and transcription termination sequences. In preferred embodiments, the vector comprises one or more nucleic acids operably encoding any one or more of the plant defensins set forth herein.

The present invention further provides host cells comprising the vectors as set forth herein. Typically, a host cell is transformed, transfected or transduced with a vector, for example, by using electroporation followed by subsequent selection of transformed, transfected or transduced cells on selective media. The resulting heterologous nucleic acid sequences in the form of vectors and nucleic acids inserted therein may be maintained extrachromosomally or may be introduced into the host cell genome by homologous recombination. Methods for such cellular transformation, transfection or transduction are well known to those of skill in the art. Guidance may be obtained, for example, from standard texts such as Sambrook et al., Molecular Cloning: A Laboratory Manual, Cold Spring Harbor, N.Y., 1989 and Ausubel et al., Current Protocols in Molecular Biology, Greene Publ. Assoc. and Wiley-Intersciences, 1992.

The present invention moreover provides expression products of the host cells as set forth herein. In some embodiments, the expression product may be polypeptides that prevent or treat proliferative diseases. In preferred embodiments, the expression product is any one or more of the plant defensins disclosed herein.

### Compositions

The present invention also provides pharmaceutical compositions for use in preventing or treating proliferative diseases, wherein the pharmaceutical compositions comprise a plant defensin, a nucleic acid, a vector, a host cell or an expression product as disclosed herein, together with a pharmaceutically acceptable carrier, diluent or excipient.

Compositions of the present invention may therefore be administered therapeutically. In such applications, compositions may be administered to a subject already suffering from a condition, in an amount sufficient to cure or at least partially arrest the condition and any complications. The quantity of the composition should be sufficient to effectively treat the patient. Compositions may be prepared according to methods which are known to those of ordinary skill in the art and accordingly may include a cosmetically or pharmaceutically acceptable carrier, excipient or diluent. Methods for preparing administrable compositions are apparent to those skilled in the art, and are described in more detail in, for example, Remington's Pharmaceutical Science, 15th ed., Mack Publishing Company, Easton, Pa., incorporated by reference herein.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes may be derived from phospholipids or other lipid substances, and may be formed by mono- or multi-lamellar hydrated liquid crystals dispersed in an aqueous medium. Any non-toxic, physiologically acceptable and metabolisable lipid capable of forming liposomes may be used. The compositions in liposome form may contain stabilisers, preservatives and excipients. Preferred lipids include phospholipids and phosphatidyl cholines (lecithins), both natural and synthetic. Methods for producing liposomes are known in the art, and in this regard specific reference is made to: Prescott, Ed., Methods in Cell Biology, Volume XIV, Academic Press, New York, N.Y. (1976), p. 33 et seq., the contents of which are incorporated herein by reference.

In some embodiments, the composition may be in the form of a tablet, liquid, lotion, cream, gel, paste or emulsion.

### Dosages

The “therapeutically effective” dose level for any particular patient will depend upon a variety of factors including the condition being treated and the severity of the condition, the activity of the compound or agent employed, the composition employed, the age, body weight, general health, sex and diet of the patient, the time of administration, the route of administration, the rate of sequestration of the plant defensin or composition, the duration of the treatment, and any drugs used in combination or coincidental with the treatment, together with other related factors well known in the art. One skilled in the art would therefore be able, by routine experimentation, to determine an effective, non-toxic amount of the plant defensin or composition which would be required to treat applicable conditions.

Typically, in therapeutic applications, the treatment would be for the duration of the disease state.

Further, it will be apparent to one of ordinary skill in the art that the optimal quantity and spacing of individual dosages of the composition will be determined by the nature and extent of the condition being treated, the form, route and site of administration, and the nature of the particular individual being treated. Also, such optimum conditions can be determined by conventional techniques.

It will also be apparent to one of ordinary skill in the art that the optimal course of treatment, such as the number of doses of the composition given per day for a defined number of days, can be ascertained by those skilled in the art using conventional course of treatment determination tests.

In terms of weight, a therapeutically effective dosage of a composition for administration to a patient is expected to be in the range of about 0.01 mg to about 150 mg per kg body weight per 24 hours; typically, about 0.1 mg to about 150 mg per kg body weight per 24 hours; about 0.1 mg to about 100 mg per kg body weight per 24 hours; about 0.5 mg to about 100 mg per kg body weight per 24 hours; or about 1.0 mg to about 100 mg per kg body weight per 24 hours. More typically, an effective dose range is expected to be in the range of about 5 mg to about 50 mg per kg body weight per 24 hours.

Alternatively, an effective dosage may be up to about 5000 mg/m². Generally, an effective dosage is expected to be in the range of about 10 to about 5000 mg/m², typically about 10 to about 2500 mg/m², about 25 to about 2000 mg/m², about 50 to about 1500 mg/m², about 50 to about 1000 mg/m², or about 75 to about 600 mg/m².

### Routes of Administration

The compositions of the present invention can be administered by standard routes. In general, the compositions may be administered by the parenteral (e.g., intravenous, intraspinal, subcutaneous or intramuscular), oral or topical route.

In other embodiments, the compositions may be administered by other enteral/enteric routes, such as rectal, sublingual or sublabial, or via the central nervous system, such as through epidural, intracerebral or intracerebroventricular routes. Other locations for administration may include via epicutaneous, transdermal, intradermal, nasal, intraarterial, intracardiac, intraosseus, intrathecal, intraperitoneal, intravesical, intravitreal, intracavernous, intravaginal or intrauterine routes.

### Carriers, Excipients and Diluents

Carriers, excipients and diluents must be “acceptable” in terms of being compatible with the other ingredients of the composition, and not deleterious to the recipient thereof. Such carriers, excipients and diluents may be used for enhancing the integrity and half-life of the compositions of the present invention. These may also be used to enhance or protect the biological activities of the compositions of the present invention.

Examples of pharmaceutically acceptable carriers or diluents are demineralised or distilled water; saline solution; vegetable based oils such as peanut oil, safflower oil, olive oil, cottonseed oil, maize oil, sesame oils, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from 10% to 99.9% by weight of the compositions.

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic acceptable diluents or carriers can include Ringer's solution, isotonic saline, phosphate buffered saline, ethanol and 1,2 propylene glycol.

### Methods for Preventing or Treating Proliferative Diseases

The present invention provides methods for preventing or treating a proliferative disease, wherein the methods comprise administering to a subject a therapeutically effective amount of a plant defensin, a nucleic acid, a vector, a host cell, an expression product or a pharmaceutical composition as disclosed herein, thereby preventing or treating the proliferative disease.

The present invention also provides use of plant defensins, nucleic acids, vectors, host cells and expression products as herein disclosed in the preparation of medicaments for preventing or treating a proliferative disease.

In some embodiments, the proliferative disease may be a cell proliferative disease selected from the group comprising an angiogenic disease, a metastatic disease, a tumourigenic disease, a neoplastic disease and cancer.

In some embodiments, the proliferative disease may be cancer. In particular embodiments, the cancer may be selected from the group comprising basal cell carcinoma, bone cancer, bowel cancer, brain cancer, breast cancer, cervical cancer, leukemia, liver cancer, lung cancer, lymphoma, melanoma, ovarian cancer, pancreatic cancer, prostate cancer or thyroid cancer.

In other embodiments, the cancer may be selected from the group comprising acute lymphoblastic leukemia, acute myeloid leukemia, adrenocortical carcinoma, AIDS-related cancers, anal cancer, appendix cancer, astrocytoma, B-cell lymphoma, basal cell carcinoma, bile duct cancer, bladder cancer, bone cancer, bowel cancer, brainstem glioma, brain tumour, breast cancer, bronchial adenomas/carcinoids, Burkitt's lymphoma, carcinoid tumour, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumour, endometrial cancer, ependymoma, esophageal cancer, extracranial germ cell tumour, extragonadal germ cell tumour, extrahepatic bile duct cancer, eye cancer, intraocular melanoma/retinoblastoma, gallbladder cancer, gastric cancer, gastrointestinal carcinoid tumour, gastrointestinal stromal tumour (GIST), germ cell tumour, gestational trophoblastic tumour, glioma, gastric carcinoid, head and/or neck cancer, heart cancer, hepatocellular (liver) cancer, hypopharyngeal cancer, hypothalamic and visual pathway glioma, Kaposi sarcoma, kidney cancer, laryngeal cancer, leukemia (acute lymphoblastic/acute myeloid/chronic lymphocytic/chronic myelogenous/hairy cell), lip and/or oral cavity cancer, liver cancer, non-small cell lung cancer, small cell lung cancer, lymphoma (AIDS-related/Burkitt/cutaneous T-Cell/Hodgkin/non-Hodgkin/primary central nervous system), macroglobulinemia, malignant fibrous histiocytoma of bone/osteosarcoma, medulloblastoma, melanoma, Merkel cell carcinoma, mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, myelogenous leukemia, myeloid leukemia, myeloproliferative disorders, nasal cavity and/or paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumour, pancreatic cancer, islet cell cancer, paranasal sinus and nasal cavity cancer, parathyroid cancer, penile cancer, pharyngeal cancer, pheochromocytoma, pineal astrocytoma, pineal germinoma, pineoblastoma and/or supratentorial primitive neuroectodermal tumours, pituitary adenoma, plasma cell neoplasia/multiple myeloma, pleuropulmonary blastoma, primary central nervous system lymphoma, prostate cancer, rectal cancer, renal cell carcinoma, retinoblastoma, rhabdomyosarcoma, salivary gland cancer, Ewing sarcoma, Kaposi sarcoma, soft tissue sarcoma, uterine sarcoma, Sezary syndrome, skin cancer (non-melanoma), skin cancer (melanoma), skin carcinoma (Merkel cell), small cell lung cancer, small intestine cancer, soft tissue sarcoma, squamous cell carcinoma, squamous neck cancer with metastatic occult primary, stomach cancer, supratentorial primitive neuroectodermal tumour, T-cell lymphoma, testicular cancer, throat cancer, thymoma and/or thymic carcinoma, thyroid cancer, transitional cancer, trophoblastic tumour, ureter and/or renal pelvis cancer, urethral cancer, uterine endometrial cancer, uterine sarcoma, vaginal cancer, visual pathway and hypothalamic glioma, vulva cancer, Waldenstrom macroglobulinemia or Wilms tumour.

### Kits

The present invention provides kits for preventing or treating a proliferative disease, wherein the kits comprise a therapeutically effective amount of a plant defensin, a nucleic acid, a vector, a host cell, an expression product or a pharmaceutical composition as herein disclosed.

The present invention also provides use of the kits disclosed herein for preventing or treating a proliferative disease, wherein the therapeutically effective amount of a plant defensin, a nucleic acid, a vector, a host cell, an expression product or a pharmaceutical composition as herein disclosed is administered to a subject, thereby preventing or treating the proliferative disease.

Kits of the present invention facilitate the employment of the methods of the present invention. Typically, kits for carrying out a method of the invention contain all the necessary reagents to carry out the method. For example, in one embodiment, the kit may comprise a plant defensin, a polypeptide, a polynucleotide, a vector, a host cell, an expression product or a pharmaceutical composition as herein disclosed.

Typically, the kits described herein will also comprise one or more containers. In the context of the present invention, a compartmentalised kit includes any kit in which compounds or compositions are contained in separate containers, and may include small glass containers, plastic containers or strips of plastic or paper. Such containers may allow the efficient transfer of compounds or compositions from one compartment to another compartment whilst avoiding cross-contamination of samples, and the addition of agents or solutions of each container from one compartment to another in a quantitative fashion.

Typically, a kit of the present invention will also include instructions for using the kit components to conduct the appropriate methods.

Methods and kits of the present invention are equally applicable to any animal, including humans and other animals, for example including non-human primate, equine, bovine, ovine, caprine, leporine, avian, feline and canine species. Accordingly, for application to different species, a single kit of the invention may be applicable, or alternatively different kits, for example containing compounds or compositions specific for each individual species, may be required.

Methods and kits of the present invention find application in any circumstance in which it is desirable to prevent or treat a proliferative disease.

### Screening for Precursors and Modulators of Compositions

The present invention provides methods for screening for cytotoxicity of plant defensins against mammalian tumour cells, wherein the method comprises contacting a plant defensin, a nucleic acid, a vector, a host cell, an expression product or a pharmaceutical composition as herein disclosed with a mammalian cell line, and assaying for cytoxicity against the mammalian cell line due to contact with the plant defensin.

The present invention also contemplates the use of nucleic acids disclosed herein and fragments or complements thereof to identify and obtain corresponding partial and complete sequences from other species using methods of recombinant DNA well known to those of skill in the art, including, but not limited to southern hybridization, northern hybridization, polymerase chain reaction (PCR), ligase chain reaction (LCR) and gene mapping techniques. Nucleic acids of the invention and fragments thereof may also be used in the production of antisense molecules using techniques known to those skilled in the art.

Accordingly, the present invention contemplates oligonucleotides and fragments based on the sequences of the nucleic acids disclosed herein for use as primers and probes for the identification of homologous sequences. Oligonucleotides are short stretches of nucleotide residues suitable for use in nucleic acid amplification reactions such as PCR, typically being at least about 10 nucleotides to about 50 nucleotides in length, more typically about 15 to about 30 nucleotides in length. Probes are nucleotide sequences of variable length, for example between about 10 nucleotides and several thousand nucleotides, for use in detection of homologous sequences, typically by hybridization. The level of homology (sequence identity) between sequences will largely be determined by the stringency of hybridization conditions. In particular, the nucleotide sequence used as a probe may hybridize to a homologue or other functionally equivalent variant of a polynucleotide disclosed herein under conditions of low stringency, medium stringency or high stringency. Low stringency hybridization conditions may correspond to hybridization performed at 50° C. in 2×SSC. There are numerous conditions and factors, well known to those skilled in the art, that may be employed to alter the stringency of hybridization. For instance, the length and nature (DNA, RNA, base composition) of the nucleic acid to be hybridized to a specified nucleic acid; concentration of salts and other components, such as the presence or absence of formamide, dextran sulfate, polyethylene glycol etc; and altering the temperature of the hybridization and/or washing steps. For example, a hybridization filter may be washed twice for 30 minutes in 2×SSC, 0.5% SDS and at least 55° C. (low stringency), at least 60° C. (medium stringency), at least 65° C. (medium/high stringency), at least 70° C. (high stringency) or at least 75° C. (very high stringency).

In preferred embodiments, the defensin is screened using an MTT (3-(4,5-Dimethylthiazol-2-yl)-2,5-diphenyltetrazolium bromide) assay. The MTT assay allows the person skilled in the art to assess the viability and proliferation of cells. Accordingly, it can be used to determine cytotoxicity of potential therapeutic agents on the basis that such agents would either stimulate or inhibit cell viability and growth. In the assay, MTT is reduced to purple formazan in living cells. A solubilization solution (usually either dimethyl sulfoxide, an acidified ethanol solution, or a solution of the detergent sodium docecyl sulfate in diluted hydrochloric acid is added to dissolve the insoluble purple formazan product into a colored solution. The absorbance of this colored solution can be quantified by measuring at a certain wavelength (usually between 500 and 600 nm) by a spectrophotometer. The absorption maximum is dependent on the solvent employed.

The present invention also provides plant defensins screened by the methods disclosed herein, for use in preventing or treating proliferative diseases.

Methods for Producing Plant Defensins with Reduced Haemolytic Activity

The present invention provides methods for producing plant defensins with reduced haemolytic activity, wherein the method comprises introducing into the plant defensin at least one alanine residue at or near the N-terminal of the defensin. The person skilled in the art would understand that several methods may be employed to achieve such addition of an N-terminal alanine, such as site-directed mutagenesis, homologous recombination, transposons and non-homologous end-joining.

Haemolytic activity may be regarded as “reduced” if the activity of the plant defensin results in relatively less hemolysis than occurs, or would reasonably be expected to occur, through use of a corresponding plant defensin that has not been modified to reduce haemolytic activity.

The present invention also provides plant defensins with reduced haemolytic activity produced by the methods disclosed herein.

### Combination Therapies

Those skilled in the art will appreciate that the polypeptides, nucleic acids, vectors, host cells, expression products and compositions disclosed herein may be administered as part of a combination therapy approach, employing one or more of the polypeptides, nucleic acids, vectors, host cells, expression products and compositions disclosed herein in conjunction with other therapeutic approaches to the methods disclosed herein. For such combination therapies, each component of the combination may be administered at the same time, or sequentially in any order, or at different times, so as to provide the desired therapeutic effect. When administered separately, it may be preferred for the components to be administered by the same route of administration, although it is not necessary for this to be so. Alternatively, the components may be formulated together in a single dosage unit as a combination product. Suitable agents which may be used in combination with the compositions of the present invention will be known to those of ordinary skill in the art, and may include, for example, chemotherapeutic agents, radioisotopes and targeted therapies such as antibodies.

Chemotherapeutic agents to be used in combination with the polypeptides, nucleic acids, vectors, host cells, expression products and compositions disclosed herein may include alkylating agents such as cisplatin, carboplatin, oxaliplatin, mechlorethamine, cyclophosphamide, chlorambucil and ifosfamide, anti-metabolites such as purine or pyramidine, plant alkaloids and terpenoids such as vinca alkaloids (including vincristine, vinblastine, vinorelbine and vindesine), and taxanes (including paclitaxel and docetaxel), podophyllotoxin, topoisomerase inhibitors such as irinotecan, topotecan, amsacrine, etoposide, etoposide phosphate and teniposide, anti-neoplastics such as doxorubicin, epirubicin and bleomycin, and tyrosine kinase inhibitors.

Targeted therapies to be used in combination with the polypeptides, nucleic acids, vectors, host cells, expression products and compositions disclosed herein may include, for example, imatinib mesylate, dasatinib, nilotinib, trastuzumab, lapatinib, gefitinib, erlotinib, cetuximab, panitumumab, temsirolimus, everolimus, vorinostat, romidepsin, bexarotene, alitretinoin, tretinoin, bortezomib, pralatrexate, bevacizumab, sorafenib, sunitinib, pazopanib, rituximab, alemtuzumab, ofatumuab, tositumomab, 131I-tositumomab, ibritumomab tiuxetan, denileukin diftitox, tamoxifen, toremifene, fulvestrant, anastrozole, exemestane and letrozole.

Other therapies may also be used in combination with the polypeptides, nucleic acids, vectors, host cells, expression products and compositions disclosed herein, including, for example, surgical intervention, dietary regimes and supplements, hypnotherapy, alternative medicines and physical therapy.

### Timing of Therapies

Those skilled in the art will appreciate that the polypeptides, polynucleotides, vectors, host cells, expression products and compositions disclosed herein may be administered as a single agent or as part of a combination therapy approach to the methods disclosed herein, either at diagnosis or subsequently thereafter, for example, as follow-up treatment or consolidation therapy as a compliment to currently available therapies for such treatments. The polypeptides, polynucleotides, vectors, host cells, expression products and compositions disclosed herein may also be used as preventative therapies for subjects who are genetically or environmentally predisposed to developing such diseases.

The person skilled in the art will understand and appreciate that different features disclosed herein may be combined to form combinations of features that are within the scope of the present invention.

The present invention will now be further described with reference to the following examples, which are illustrative only and non-limiting.

### EXAMPLES

### Materials and Methods

Purification of NaD1 from Nicotiana alata

To isolate NaD1 from its natural source, whole N. alata flowers up to the petal coloration stage of flower development were ground to a fine powder and extracted in dilute sulfuric acid as described previously (Lay et al., 2003a). Briefly, flowers (760 g wet weight) were frozen in liquid nitrogen, ground to a fine powder in a mortar and pestle, and homogenized in 50 mM sulfuric acid (3 mL per g fresh weight) for 5 min using an Ultra-Turrax homogenizer (Janke and Kunkel). After stirring for 1 h at 4° C., cellular debris was removed by filtration through Miracloth (Calbiochem, San Diego, Calif.) and centrifugation (25,000×g, 15 min, 4° C.). The pH was then adjusted to 7.0 by addition of 10 M NaOH and the extract was stirred for 1 h at 4° C. before centrifugation (25,000×g, 15 min, 4° C.) to remove precipitated proteins. The supernatant (1.8 L) was applied to an SP Sepharose™ Fast Flow (GE Healthcare Bio-Sciences) column (2.5×2.5 cm) pre-equilibrated with 10 mM sodium phosphate buffer. Unbound proteins were removed by washing with 20 column volumes of 10 mM sodium phosphate buffer (pH 6.0) and bound proteins were eluted in 3×10 mL fractions with 10 mM sodium phosphate buffer (pH 6.0) containing 500 mM NaCl. Samples from each purification step were analyzed by SDS-polyacrylamide gel electrophoresis (SDS-PAGE) and immunoblotting with the anti-NaD1 antibodies. Fractions from the SP Sepharose column containing NaD1 were subjected to reverse-phase high performance liquid chromatography (RP-HPLC).

### Reverse-Phase High Performance Liquid Chromatography

Reverse-phase high performance liquid chromatography (RP-HPLC) was performed on a System Gold HPLC (Beckman) coupled to a detector (model 166, Beckman) using a preparative C8 column (22×250 mm, Vydac) with a guard column attached. Protein samples were loaded in buffer A (0.1% [v/v] trifluoroacetic acid) and eluted with a linear gradient of 0-100% (v/v) buffer B (60% [v/v] acetonitrile in 0.089% [v/v] trifluoroacetic acid) at a flow rate of 10 mL/min over 40 min. Proteins were detected by monitoring absorbance at 215 nm (FIG. 1B). Protein peaks were collected and analyzed by SDS-PAGE.

Samples from each stage of NaD1 purification (30 μL) were added to NuPAGE® (Registered Trademark) LDS sample loading buffer (10 μL, Invitrogen) and heated to 70° C. for 10 min. The samples were then loaded onto NuPAGE® precast 4-12% Bis-Tris polyacrylamide gels (Invitrogen) and the proteins were separated using an XCell-Surelock electrophoresis apparatus (Invitrogen) run at 200 V. Proteins were visualized by Coomassie Blue staining or transferred onto nitrocellulose for immunoblotting with the anti-NaD1 antibodies.

Isolation of Other Defensins from Plants (NsD1, NsD2, PhD1a)

Defensins were isolated from seeds or flowers using the procedure described herein for purification of NaD1 from Nicotiana alata flowers. Briefly, seeds (500 g) were placed in an Ultra-Turrax homogenizer (Janke and Kunkel) and ground to a fine powder before addition of 50 mM sulfuric acid (4 mL per g fresh weight). Flowers were ground to a fine powder in liquid nitrogen before the addition of 50 mM sulfuric acid (3 mL per g fresh weight). Homogenisation was continued for 5 min before the homogenate was transferred to a beaker and stirred for 1 h at 4° C. Cellular debris was removed by filtration through Miracloth (Calbiochem, San Diego, Calif.) and centrifugation (25,000×g, 15 min, 4° C.). The pH was then adjusted to 7.0 by addition of 10 M NaOH and the extract was stirred for 1 h at 4° C. before centrifugation (25,000×g, 15 min, ° C.) to remove precipitated proteins. The supernatant was applied to an SP-Sepharose™ Fast Flow (GE Healthcare Bio-Sciences) column (2.5×2.5 cm) pre-equilibrated with 10 mM sodium phosphate buffer. Unbound proteins were removed by washing with 20 column volumes of 10 mM sodium phosphate buffer (pH 6.0) and bound proteins were eluted in 3×10 mL fractions with 10 mM sodium phosphate buffer (pH 6.0) containing 500 mM NaCl.

Fractions from the SP Sepharose column were subjected to reverse-phase high performance liquid chromatography (RP-HPLC) using either an analytical Zorbax 300SB-C8 RP-HPLC column and an Agilent Technologies 1200 series system or a preparative Vydac C8 RP-HPLC column on a Beckman Coulter System Gold HPLC. Protein samples were loaded in buffer A (0.1% (v/v) trifluoroacetic acid) and eluted with a linear gradient of 0-100% (v/v) buffer B (60% (v/v) acetonitrile in 0.089% (v/v) trifluoroacetic acid. Eluted proteins were detected by monitoring absorbance at 215 nm. Protein peaks were collected and defensins were identified using SDS-PAGE and mass spectrometry.

Expression and Purification of Recombinant Defensins in Pichia pastoris

The Pichia pastoris expression system is well-known and commercially available from Invitrogen (Carlsbad, Calif.; see the supplier's Pichia Expression Manual disclosing the sequence of the pPIC9 expression vector). The defensins of interest, including NaD1, TPP3, γ2-z, γ1-H, Dm-AMP1 were cloned into the pPIC9 expression vector (the proteins encoded by these clones were designated rNaD1, rTPP3, rγ2-z, rγ1-H, rDm-AMP1, respectively). These constructs were then used to transform P. pastoris GS115 cells. A colony of each clone was used to inoculate 10 mL of BMG medium (described in the Invitrogen Pichia Expression Manual) in a 100 mL flask and was incubated overnight in a 30° C. shaking incubator (140 rpm). The culture was used to inoculate 500 mL of BMG in a 2 L baffled flask which was placed in a 30° C. shaking incubator (140 rpm). Once the OD₆₀₀ reached 2.0 (˜18 h), cells were harvested by centrifugation (2,500×g, 10 min) and resuspended into 1 L of BMM medium (OD₆₀₀=1.0) in a 5 L baffled flask and incubated in a 28° C. shaking incubator for 3 days. The expression medium was separated from cells by centrifugation (4750 rpm, 20 min) and diluted with an equal volume of 20 mM potassium phosphate buffer (pH 6.0). The medium was adjusted to pH 6.0 with NaOH before it was applied to an SP Sepharose column (1 cm×1 cm, Amersham Biosciences) pre-equilibrated with 10 mM potassium phosphate buffer, pH 6.0. The column was then washed with 100 mL of 10 mM potassium phosphate buffer, pH 6.0 and bound protein was eluted in 10 mL of 10 mM potassium phosphate buffer containing 500 mM NaCl (FIG. 1A). Eluted proteins were subjected to RP-HPLC using a 40 minute linear gradient as described herein below. Protein peaks were collected and analyzed by SDS-PAGE and immunoblotting with the anti-NaD1 antibody. Fractions containing the defensin were lyophilized and resuspended in sterile milli Q ultrapure water. The protein concentration of Pichia-expressed defensin was determined using the bicinchoninic acid (BCA) protein assay (Pierce Chemical Co.) with bovine serum albumin (BSA) as the protein standard.

Circular Dichroism Spectrum of rNaD1

To examine whether NaD1 purified from P. pastoris (rNaD1) was correctly folded, its far UV circular dichroism (CD) spectrum was recorded and compared with that of native NaD1 (FIG. 1C). The similarity of the two spectra indicates the structure of rNaD1 was not significantly altered compared to native NaD1.

Antifungal Activity of rNaD1

The effect of rNaD1 on the growth of Fusarium oxysporum f. sp. vasinfectum was compared to that of native NaD1. Recombinant NaD1 demonstrated antifungal activity at low concentrations with an IC₅₀ of ˜1.6 μM. NaD1 was slightly more effective with an IC₅₀ of ˜1.0 μM (FIG. 1D).

### Preparation of Reduced and Alkylated NaD1

Lyophilized NaD1 (500 μg) was dissolved in 400 μL of stock buffer (200 mM Tris-HCl pH 8.0, 2 mM EDTA, 6 M guanidine-HCl, 0.02% [v/v] Tween®-20). Reduction buffer (stock buffer with 15 mM dithiothreitol [DTT]) was added (44 μL) followed by a 4.5 h incubation at 40° C. The reaction mixture was cooled to RT before iodoacetic acid (0.5 M in 1 M NaOH, 55 μL) was added and the incubation continued in the dark for 30 min at RT. A Nanosep Omega® (Registered Trademark) spin column (3K molecular weight cut off, PALL Life Sciences) was used to remove salts, DTT and iodoacetic acid and the protein concentration was determined using the BCA protein assay (Pierce). The effect of reduced and alkylated NaD1 (NaD1_{R&A}) on the growth of Fov was measured as described herein.

### Immunoblot Analysis

For immunoblot analysis, proteins were transferred to nitrocellulose and probed with protein A-purified anti-NaD1 antibodies (1:3000 dilution of 7.5 mg/mL) followed by goat anti-rabbit IgG conjugated to horseradish peroxidase (1:3500 dilution; Amersham Pharmacia Biotech). Enhanced chemiluminescence (ECL) detection reagents (Amersham Pharmacia Biotech) were used to visualize bound antibodies with a ChemiGenius™ bioimaging system (Syngene).

To produce anti-NaD1 or anti-NaD2 antiserum, purified NaD1 or NaD2 (1.5 mg) were conjugated to Keyhole Limpet Hemocyanin (0.5 mg, Sigma) respectively, with glutaraldehyde as described by Harlow and Lane (1988). A rabbit was injected with 1.5 mL of protein (150 μg NaD1) in an equal volume of Freund's complete adjuvant (Sigma). Booster immunizations of conjugated protein (100 μg NaD1 or NaD2) and Freund's incomplete adjuvant (Sigma-Aldrich) were administered four and eight weeks later. Pre-immune serum was collected before injection and immune serum was collected 14 d after the third and fourth immunizations. The IgG fraction from both pre-immune and immune serum was purified using Protein-A Sepharose CL-4B (Amersham Pharmacia Biotech) and was stored at −80° C. at concentrations of 3.4 mg/mL and 7.5 mg/mL, respectively.

Bacterial Expression and Purification of rStPin1A

The type I serine proteinase inhibitor StPinIA, isolated from potato (Solanum tuberosum) was previously described (as Pot1A) in U.S. Pat. No. 7,462,695 “Insect chymotrypsin and inhibitors thereof” and 11/753,072 “Multi-Gene Expression Vehicle” and is incorporated herein by reference.

The DNA fragment encoding the mature domain of StPin1A was PCR-amplified for subcloning into the vector pHUE for recombinant protein expression in E. coli (Baker et al, 2005, Cantanzariti et al, 2004). The following primers were used: Sac2StPin1A5′: 5′ CTC CGC GGT GGT MG GAA TCG GAA TCT GAA TCT TG 3′; PotISalI3′: 5′ GGT CGA CTT AAG CCA CCC TAG GM TTT GTA CM CAT C 3′, which incorporated Sac II and Sal I restriction sites at the 5′ and 3′ ends respectively. PCR reactions contained 2× GoTaq Mastermix (25 μL, Promega), Sac2PotI5′ primer (10 μM, 2 μL), PotISalI3′ primer (10 μM, 2 μL), sterile distilled water (16 μL) and pGEM-T Easy-StPin1A plasmid DNA (−20 ng, 5 μL) as template. Initial denaturing occurred at 94° C. for 2 min, followed by 30 cycles of 94° C. for 1 min, 60° C. for 1 min and 72° C. for 1 min followed by a final elongation step of 72° C. for 10 min.

The PCR product was cloned into the pCR2.1-TOPO vector (Invitrogen) which was then used to transform chemically competent E. coli TOP10 cells (Invitrogen) according to the manufacturers instructions. Plasmid DNA was isolated using the Wizard Plus SV Miniprep kit (Promega) and vector inserts were sequenced (Macrogen) using the TOPO-specific M13 forward and reverse primers.

Inserts were excised using Sac II and Sal I, extracted from agarose gels using the Perfectprep kit (Eppendorf) and ligated into pHUE which was then used to transform E. coli TOP10 cells. Plasmid DNA for pHUE containing StPin1A was isolated and then used to transform E. coli BL21 (DE3) CodonPlus-RIL cells (Stratagene).

Single colonies of transformed E. coli were used to inoculate 20 mL of 2YT media (10 mL, 16 g/L tryptone, 10 g/L yeast extract, 5 g/L NaCl) containing ampicillin (0.1 mg/mL), chloramphenicol (0.034 mg/mL) and tetracycline (0.01 mg/mL) and grown overnight with shaking at 37° C. This culture was used to inoculate fresh 2YT media (1 L) containing antibiotics which was then incubated at 37° C. with shaking until an optical density (595 nm) of ˜0.8. IPTG was added (1 mM final concentration) and the culture grown for a further 3 h.

The cells were harvested by centrifugation and the soluble recombinant protein was purified by affinity chromatography on nickel-nitrilotriacetic acid (Ni-NTA) resin (Qiagen) using the native protein purification protocol outlined in The QiaExpressionist Manual (Qiagen). Bound protein was eluted from the resin in a buffer containing 250 mM imidazole before dialysis for 8-16 h at 4° C. in a solution containing 50 mM Tris-HCl (pH 8.0) and 300 mM NaCl. The dialyzed fusion protein was cleaved by incubation with the de-ubiquitylating protease, 6H.Usp2-cc (Catanzariti et al., 2004; Baker et al., 2005) for 1 h at 37° C. The cleaved protein was subsequently purified using a System Gold HPLC (Beckman) coupled to a detector (model 166, Beckman) and a preparative C8 column (22×250 mm, Vydac). Protein samples were loaded in buffer A (0.1% [v/v] trifluoroacetic acid) and eluted with a step gradient of 0-60% (v/v) buffer B (60% [v/v] acetonitrile in 0.089% [v/v] trifluoroacetic acid) over 5 min and 60-100% buffer B over 20 min with a flow rate of 10 ml/min. Proteins were detected by monitoring absorbance at 215 nm. Protein peaks were collected manually and analyzed by SDS-PAGE.

### Cell Lines and Culture

Mammalian cell lines used in this study were as follows: human melanoma cancer MM170 cells, immortalized T lymphocyte Jurkat cells, human leukemia monocyte lymphoma U937 cells, human prostate cancer PC3 cells, mouse melanoma B16 cells, Chinese hamster ovary (CHO) cells, GAG-deficient CHO mutant pgsA-745 cells, and African green monkey kidney fibroblast COS-7 cells. The cells were grown in tissue culture flasks at 37° C. under a humidified atmosphere of 5% CO₂/95% air, and sub-cultured routinely two to three times a week according to the rate of proliferation. All mammalian cells were cultured in RPMI-1640 medium (Invitrogen) supplemented with 10% heat-inactivated fetal bovine serum (FBS, Invitrogen), 100 U/mL penicillin (Invitrogen) and 100 μg/mL streptomycin (Invitrogen), with the exception that CHO and PGS cells were cultured in DMEM-F12 medium (DMEM, Invitrogen) supplemented with 10% FBS, 100 U/mL penicillin and 100 μg/mL streptomycin. Adherent cell lines were detached from the flask by adding 3-5 mL of a mixture containing 0.25% trypsin and 0.5 μM EDTA (Invitrogen).

### Ficoll-Paque Isolation of Peripheral Blood Mononuclear Cells (PBMCs)

PBMCs were re-suspended to a cell concentration of 1×10⁶ PBMCs/mL, following ficoll-paque isolation. Briefly, blood was collected in heparinised tubes, and diluted 1 in 2 with sterile 1×PBS/0.5% BSA (D-PBS, Ca²⁺ and Mg²⁺ free, Invitrogen). Using sterile 50 mL tubes, diluted blood (35 mL) was over-laid on 15 mL ficoll-paque, followed by centrifugation for 30 min at 1800 rpm (break off). The upper plasma layer was removed into a fresh tube and re-spun, prior to removing PBMC layer and dividing cells between four tubes topped with 1×PBS/0.5% BSA. Cells were spun for 10 min at 1000 rpm RT with the pellet of each tube washed (×3) with 50 mL 1×PBS/0.5% BSA. To remove more platelets, cells were spun for 15 min at 800 rpm.

### Red Blood Cell (RBC) Lysis

Following ficoll-paque separation, RBCs were collected and washed with 1×PBS and pelleted at 1000×g for 10 min. RBCs were diluted 1 in 10 for treatment with increasing concentrations (0-100 μM) of defensins and incubated over-night under a humidified atmosphere of 5% CO₂/95% air. Post 24 h incubation, the cells were centrifuged for 10 min at 2000 rpm, with the supernatant diluted to 1 in 100 with 1×PBS. The degree of red blood cell lysis was measured as absorbance at 412 nm.

### MTT Cell Viability Assays

Tumour cells were seeded in quadruplicate into wells of a flat-bottomed 96-well microtitre plate (50 μL) at various densities starting at 2×10⁶ cells/mL. Four wells containing complete culture medium alone were included in each assay as a background control. The microtitre plate was incubated overnight at 37° C. under a humidified atmosphere containing 5% CO₂/95% air, prior to the addition of complete culture medium (100 μL) to each well and further incubated at 37° C. for 48 h. Optimum cell densities (30-50% confluency) for cell viability assays were determined for each cell line by light microscopy.

Tumour cells were seeded in a 96-well microtitre plate (50 μL/well) at an optimum density determined in the cell optimisation assay as above. Background control wells (n=8) containing the same volume of complete culture medium were included in the assay. The microtitre plate was incubated overnight at 37° C., prior to the addition of proteins at various concentrations and the plate was incubated for a further 48 h. The cell viability 3-(4,5-dimethyl-2-thiazolyl)-2,5-diphenyl-2H-tetrazolium bromide (MTT, Sigma-Aldrich) assay was carried out as follows: the MTT solution (1 mg/mL) was added to each well (100 μL) and the plate incubated for 2-3 h at 37° C. under a humidified atmosphere containing 5% CO₂/95% air. Subsequently, for adherent cell lines, the media was removed and replaced with dimethyl sulfoxide (100 μL, DMSO, Sigma-Aldrich), and placed on a shaker for 5 min to dissolve the tetrazolium salts. In the case of suspension cells, prior to the addition of DMSO the cells are spun at 1500 rpm for 5 min. Absorbance of each well was measured at 570 nm and the IC₅₀ values (the protein concentration to inhibit 50% of cell growth) were determined using the Origin Software Program.

### ATP Bioluminescence Assay

ATP bioluminescence assay (Roche Diagnostics, NSW Australia) was used to quantitate the release of ATP by permeabilised tumour cells. The Luciferase reagent was dissolved as per manufacturer's instructions and incubated for 5 min at 4° C. Briefly, cells were re-suspended at a concentration of 1×10⁶ cells/mL in 1×PBS/0.1% BSA and added (40 μL/well) to the Luciferase reagent (50 μL/well) to a blank microtitre plate (Nunc™) containing 10 μL of protein samples. Simultaneously, using a multichannel pipette, the mixture was added (90 μL/well) and samples were immediately read on a microtitre plate reader at 562 nm for 30 min with readings taken at 30 s intervals. The data were analysed by SoftMaxPro 4.0 software (Molecular Devices Company).

### Fluorescent Activated Cell Sorting (FACS) Cell Permeability Assay

Unless otherwise stated, cells were re-suspended at a cell concentration of 4×10⁵ cells/mL in complete culture RPMI-1640 medium supplemented with 10% FBS, 100 U/mL penicillin and 100 μg/mL streptomycin, and added to either a V-bottom 96-well plate or microfuge tubes. Cells were kept at 37° C., unless otherwise stated, during protein addition (5 μL) at various concentrations or the set concentration of 10 μM. Typically, cells were mixed with the protein of interest and incubated at 37° C. for 30 min. In certain experiments, cells were also incubated at either 4° C. or 37° C. for 2-60 min prior to flow cytometry analysis. Cells were added to an equal volume of complete culture medium containing 2 μg/mL propidium iodide (PI, Annexin V-FITC Apoptosis Detection Kit, Invitrogen) and analysed immediately by flow cytometry using a FACSCanto cell sorter (Becton Dickson, Fanklin Lakes, N.J.) and Cell Quest Pro Software (Becton Dickson). Typically, 5000-10000 events per sample were collected and the resultant data were analysed using FlowJo software (Tree Star, Ashland, Oreg.). Cells were gated appropriately based on forward scatter (FSC) and side scatter (SSC), with the viable cells determined by their ability to exclude PI. For analysis purposes, all data was standardised relative to control (normal cell % ranged from approx. 0-7%).

### Scanning Electron Microscopy of Permeabilised Cells

Scanning electron microscopy was used in this study to visualize PC3 cells when treated with NaD1 (10 μM) in comparison to untreated control. Once removed from the incubator the cells were kept on ice until required. Small glass petri dishes were layered with filter paper soaked in distilled water, and the cover-slips were later laid onto the dish. The samples were washed with the wash buffer (0.2 M sodium phosphate (pH 7.2) and 5.4% (w/v) glucose) prior to primary fixation, samples were immersed in equal parts of 1.25% glutaraldehyde and 0.5% osmium tetroxide fixative for 30 min at 4° C. The samples were washed twice for 15 min with wash buffer, followed by immersion in 2% osmium for 1 h on ice and in a light-/air-tight glass petri dish. The samples were then washed three times for 5 min with wash buffer prior to the subsequent dehydration procedure. The dehydration step in the protocol was then carried out and required sequential immersion in increasing concentrations of ethanol (EtOH): 1×10 min in 50% EtOH, 1×10 min in 70% EtOH, 1×10 min 90% EtOH, 1×10 min in 95% EtOH, and finally 2×10 min in 100% EtOH. The fixing and dehydrating of samples was followed by Freeze Drying, where the samples were immersed for a few sec in melting nitrogen then placed in a copper block in a vacuum evaporator (Dynavac). Following 48 h of freeze drying, the sample is mounted onto a metal stub and stored in a desiccator. The samples were finally coated with a thin layer of metals (gold and palladium) using an automated sputter coater (SC7640 Polaron). Samples were analysed using a high resolution digital Field Emission-Scanning Electron Microscope, FE-SEM (JSM-6340F, JEOL Ltd, Japan).

### Lipid-Coated Membrane Strip-Based Assay

Membrane Lipid Strips™, PIP Strips™ and Sphingo Strips™ (Echelon Biosciences, Salt Lake City, Utah) were incubated with PBS/3% BSA for 1-2 h at RT to block non-specific binding. The membrane strips were then incubated with defensins (0.12 μM) diluted in PBS/1% BSA overnight at 4° C., prior to thorough washing for 60 min at RT with PBS/0.1% Tween-20. Membrane-bound protein was detected by probing the membrane strips with a rabbit anti-NaD1 polyclonal antibody (for detection of NaD1, NsD1, NsD2, rTPP3 or PhD1A) or a rabbit anti-NaD2 antibody (for detection of NaD2 or NsD3) (in both cases diluted 1:2000 with PBS/1% BSA) for 1 h at 4° C., followed by a HRP-conjugated donkey anti-rabbit IgG antibody (diluted 1:2000 with PBS/1% BSA) for 1 h at 4° C. After each antibody incubation, the membrane strips were washed extensively for 60 min at RT with PBS/0.1% Tween-20. Chemiluminescence was detected using the enhanced chemiluminescence (ECL) western blotting reagent (GE Healthcare BioSciences, NSW Australia) and exposed to Hyperfilm (GE Healthcare BioSciences, NSW, Australia) and developed using an Xomat (All-Pro-Imaging).

Densitometry analysis was performed on images obtained from lipid strips using ImageJ (National Institute of Health, Bethesda, Md.). Briefly, circles of equivalent size were traced around areas of interest. A background circle of equal size was also placed in the area on the membrane where there is no lipid and set as the background. The areas of interest were quantified as the average pixel intensity subtracted from the background.

### Example 1

### In Vitro Anti-Tumour Activity of NaD1

### Example 1

### Introduction

The effect of NaD1 (either purified native protein from the flowers of N. alata or purified recombinant protein produced in P. pastoris) on the viability of tumour cell lines and primary human cell isolates was determined using a 3-(4,5-dimethyl-2-thiazolyl)-2,5-diphenyl-2H-tetrazolium bromide (MTT) in vitro cell culture viability assay. The tumour cell lines tested were HCT116 (human colon cancer), MCF-7 (human breast cancer), MM170 (human melanoma), PC3 (human prostate cancer), B16-F1 (mouse melanoma), CASMC (human coronary artery smooth muscle cells) and HUVEC (human umbilical vein endothelial cells). NaD1 was tested alongside the purified plant proteins recombinant StPin1A (rStPin1A) or NaD2. Cells were seeded into 96-well flat-bottomed microtitre plates at the following cell numbers: MM170 (2×10⁴/well), MCF-7 (2×10⁴/well), HCT-116 (5×10³/well), PC3 (5×10³/well), B16-F1 (2×10³/well), HUVEC (3×10³/well), CASMC (5×10³/well) and cultured overnight. NaD1, rNaD1 or rStPin1A were then added to cells to final concentrations ranging from 1 to 100 μM and incubated for 48 h, upon which MTT assays were carried out as described in the Materials and Methods.

### Example 1

### Results

NaD1 and rNaD1 dramatically decreased the viability of all the tumour cell lines tested with IC₅₀ values at low μM concentrations (2 to 50) (FIGS. 2A to 2E). Both forms of NaD1 showed very similar inhibitory effects, with NaD1 having only slightly greater activity than rNaD1 (FIG. 2F). In contrast, the plant protein rStPin1A showed no significant effect on cell viability of the tumour cells lines (FIGS. 2A to 2E). NaD2 (a solanaceous Class I defensin also isolated from the flowers of N. alata) was also tested on MM170 cells and no significant effect on cell viability was observed (FIG. 2G). NaD1 and rNaD1 were also found to reduce the cell viability of normal human CASMC and HUVEC but the IC₅₀ values were higher (7.5 to 12 μM) than that for the tumour cells lines (FIGS. 2H and 2I). NaD2 or rStPin1A showed no significant effect on cell viability of CASMC and HUVEC (FIGS. 2H and 2I). In comparison to NaD1, a reduced and alkylated form of NaD1 (NaD1_{R&A}) showed no effect on tumour cell viability when tested on the mouse melanoma B16-F1 (FIG. 2J). These data indicate that NaD1 in both native and recombinant forms selectively kills tumour cells at low μM concentrations.

### Example 2

### Effect of NaD1 on the Permeabilisation of Human Cell In Vitro

### Example 2

### Introduction

NaD1 has previously been shown to have the ability to permeabilise the hyphae of F. oxysporum f. sp. vasinfectum (van-der-Weerden et al, 2008). To determine whether NaD1 kills tumour cells in a similar manner to fungus, the ability of NaD1 to permeabilise tumour cells was assessed using two different approaches. The first used a bioluminescence assay to measure the release of intracellular ATP. 4×10⁴ U937 (human myelomonocytic tumour cell line) or MM170 (human myeloma) cells were treated with increasing concentrations of native NaD1 (0-20-μM) and ATP release measured at intervals of 30 seconds for a total of 30 min by determining absorbance at 562 nm. The second approach used flow cytometry to determine the uptake of the fluorescent dye propidium iodide (PI) (2 mg/mL) by U937 and MM170 cells (4×10⁵/mL) following the treatment of cells with increasing concentrations of NaD1 (0 to 100 μM) for 30 min.

In addition, field emission-scanning electron microscopy (FE-SEM) was used to observe any morphological changes to the tumour cell membrane. Human PC3 cells (prostate cancer were treated with NaD1 (10 μM) or not for 30 min and subsequently fixed and processed for FE-SEM.

### Example 2

### Results

U937 and MM170 cells showed a release of ATP in a time-dependent and concentration-dependent manner when treated with NaD1 (FIGS. 3C and 3D). In both cases ATP was released from cells almost immediately upon exposure to NaD1. NaD1_{R&A} showed no ability to permeabilise U937 or MM170 (FIG. 3D). These results indicate that the intact structure of NaD1 is essential for cell permeabilisation and correlate with ability of NaD1 to kill tumour cells as indicated in Example 2.

To further examine tumour cell permeabilisation by NaD1, U937 and MM179 cells were treated with increasing concentrations of NaD1 (0 to 100 μM) for 30 min at 37° C. and then PI uptake measured by flow cytometry. As described for the release of ATP mediated by NaD1, the uptake of PI by both U937 and MM170 cells increased with increasing concentrations of NaD1. As shown in FIGS. 3A and B the number of PI⁺ U937 or MM170 cells was similar upon exposure to different concentrations of NaD1, with ˜30% PI⁺ at 6.25 μM which increased to 100% PI⁺ at 100 μM.

The examination of PC3 cells that were exposed to NaD1 by FE-SEM indicated that they showed a clear morphological difference to untreated cells. NaD1 treated cells exhibited a disrupted plasma membrane as demonstrated by the distorted irregular cell surface in comparison to the smooth intact surface of untreated cells (FIG. 3E). These changes are indicative of a destabilised plasma membrane and support the findings described above that NaD1 permeabilises the plasma membrane of tumour cells.

### Example 3

### Effect of NaD1 and Recombinant NaD1 on Red Blood Cell Lysis

### Example 3

### Introduction

The ability of native NaD1 or rNaD1 to lyse human red blood cells (RBCs) was investigated by incubating 10⁷ RBCs with increasing concentrations of NaD1 (0 to 100 μM) for 16 h at 37° C. and determining haemoglobin release by measuring absorbance at 412 nm.

### Example 3

### Results

NaD1 at low concentrations (<12.5 μM) had no effect on RBC lysis when compared to the PBS only control. However, a higher concentration of NaD1 (12.5 to 100-μM) did induce RBCI lysis, with the levels of released haemoglobin reaching a maximum of ˜50% lysis at 100 μM (relative to the positive control whereby lysis was induced to 100% completion with water). In contrast, rNaD1 showed no ability to lyse RBCs at concentrations up to 100 μM (FIG. 4).

### Example 4

### Permeabilisation Activity of NaD1 in the Presence of Serum

### Example 4

### Introduction

To assess the ability of NaD1 to permeabilise tumour cells in the presence of serum, the PI-uptake flow cytometry assay was utilised as described in Example 2 with the following modifications: 4×10⁵/mL U937 cells were incubated with 10 μM native NaD1 in the presence of increasing concentrations of foetal calf serum (FCS) (0 to 40%) for 60 min followed by addition of 2 mg/mL PI. The percentage of PI⁺ cells was then determined by flow cytometry.

### Example 4

### Results

NaD1 retained the ability to permeabilise U937 cells in the presence of serum as demonstrated by the detection of 70% PI⁺ cells in the presence of 40% FCS, which was only marginally lower than the 90% PI⁺ cells at 0% FCS.

### Example 5

### In Vivo Anti-Tumour Activity of NaD1

### Example 5

### Introduction

The effect of NaD1 on tumour growth was assessed in an in vivo model of solid melanoma growth in mice. C57BL/6 mice were injected subcutaneously with 5×10⁵ B16-F1 tumour cells and solid tumours grown to a diameter of ˜10 mm. One mg/kg body weight NaD1 or NaD1_{R&A} in 504 of PBS, or 50 μL of PBS alone was then injected intratumuorally every 2 days until mice were sacrificed. The tumour size was measured before injection every 2 days. Six mice were used in each group.

### Example 5

### Results

The intratumour injection of 1 mg/kg body weight NaD1 resulted in a significant reduction in tumour growth when compared to the controls of NaD1_{R&A} and PBS alone. By day 4 the average tumour size had reached only 1.8±0.2 for NaD1 treated mice compared to 4.0±0.4 or 3.7±0.6 for NaD1_{R&A} or PBS alone treated mice, respectively (tumour size was normalised to 1 for each mouse at day 0). It should be noted that the B16-F1 tumours were established at a highly advanced stage when treatment was initiated.

### Example 6

### Acute Oral Toxicity Testing of NaD1 in Mice

### Example 6

### Introduction

This study was based on OECD Test Guideline 423 (OECD [Organisation for Economic Co-operation and Development]. 2001. Guideline 423: Acute Oral Toxicity—Acute Toxic Class Method. Paris: OECD).

Healthy female C57BL/6 mice derived from the same litter were obtained from either the Central Animal House at La Trobe University (Bundoora Campus) or from Monash Animal Services. The animals were identified by ear punch and kept three per cage during the study. The animals will be housed and maintained in groups of three in cages as per standard animal house conditions at La Trobe University.

On the day of dosing, the test mice were weighed and fasted for 4 h prior to dose administration. Just prior to dosing, the mice were reweighed. The protein solution (pure NaD1 in water) was prepared shortly prior to administration such that each of the three test mice received a total of 400 μL of the protein solution at the fixed dosing level of either 0 (water only vehicle control), 20, 50 or 300 mg NaD1/kg body weight. The protein solution was administered by oral gavage using a round-tipped canula needle. Feed was replaced 1 h after dosing. The mice received standard rodent diet and water ad libitum.

The mice were observed hourly for 4 h after dosing on day 1 and at least twice daily thereafter until scheduled killing on day 14. Signs of gross toxicity, adverse pharmacologic effects and behavioural changes were assessed and recorded daily as was the food and water consumption. The mice were reweighed at days 7 or 8 and 14. On the last day of the study (day 14), the mice were killed by inhalation of carbon dioxide and necropsied. All the mice received a gross pathological examination. The weights of the following organs were recorded: brain, heart, liver, lungs, kidneys, gastrointestinal tract, spleen and thymus. Subsequently, the samples were fixed in 4% (v/v) paraformaldehyde until paraffin embedding, sectioning and histopathological examination by the Australian Phenomics Network, University of Melbourne node. The gastrointestinal tract was divided into the following sections: stomach, duodenum, jejunum, ileum, cecum and colon.

### Example 6

### Results: Bodyweights and Clinical Signs

All animals appeared healthy, showed no signs of gross toxicity, adverse pharmacologic effects or behavioural changes and survived to termination of the study. There was no treatment related effects on body weight, with weights closely matching that of the pre-fast weight at the commencement of the study.

At the end of the study, the mice were killed by carbon dioxide asphyxiation and the organs were the following organs were collected: brain, heart, liver, lungs, kidneys, gastrointestinal tract, spleen and thymus. These tissues were fixed in 4% (v/v) paraformaldehyde. The gastointestinal tract was subsequently divided into the following sections: stomach, duodenum, jejunum, ileum, cecum and colon. All the organs were embedded in paraffin, sectioned and stained with hematoxylin and eosin (and Luxol fast blue for brain sections) by the Australian Phenomics Network, University of Melbourne node.

No pathologies, attributable to protein administration, were observed in any of the mice except for possible slight irritation to the stomach epithelium at the highest dose of 300 mg NaD1/kg body weight.

### Example 7

### Cellular Lipid Binding Properties of NaD1 and NaD2

### Example 7

### Introduction

The interaction of NaD1 and NaD2 to cellular lipids was tested by performing solid-state lipid binding assays using three different commercially available lipid strips from Echelon™ (Membrane, PIP, and Sphingolipid Strips). These strips are spotted with 100 pmole of each lipid in a biologically active form. NaD1, or NaD2, rNaD1 or rNaD2 (0.12 μM) were incubated overnight at 4° C. with the lipid strips and binding detected with specific rabbit polyclonal antibodies to NaD1 or NaD2 followed by a HRP-conjugated donkey anti-rabbit antibody. NaD1 or NaD2 binding was quantitated by carrying out densitometry on the developed lipid strips.

### Example 7

### Results

NaD1 bound most strongly to the phosphoinositides PtdIns (PIP₂) and (PIP₃) including PtdIns(3,5)P₂, PtdIns(3,5)P₂, PtdIns(4,5)P₂ and PtdIns(3,4,5)P₃, but also showed strong binding to cardiolipin and the PtdIns(PIP) including PtdIns(3)P, PtdIns(4)P₂, and PtdIns(5)P (FIGS. 7A and 7B). NaD1 also showed weak binding to the phosphatidylserine, phosphatidylalanine, phosphatidylglycerol, and sulfatide (FIGS. 7A, B and C). Recombinant NaD1 showed a similar lipid binding specificity to NaD1, with the exception that stronger binding was observed to phosphatidylserine, phosphatidylalanine and phosphatidylglycerol (FIG. 7G). NaD1_{R&A} showed no binding to any cellular lipids (FIG. 7G).

NaD2 was also found to bind cellular lipids but with a specificity distinct to that of NaD1. In contrast to NaD1, NaD2 showed strong binding to phosphatidic acid, but no apparent binding to PtdIns(3,5)P₂, PtdIns(3,5)P₂, PtdIns(4,5)P₂ and PtdIns(3,4,5)P₃.

However like NaD1, NaD2 also showed binding to PtdIns(3)P, PtdIns(4)P, and PtdIns(5)P₂ (FIGS. 7D, E and F). Collectively, these data suggest that the related defensins NaD1 and NaD2 both bind cellular phospholipids with overlapping but different specificities. Recombinant NaD2 showed a similar lipid binding specificity to NaD2, with the exception that stronger binding was observed to phosphatidylserine (FIG. 7G). In contrast to rNaD1, rNaD2 showed no lipid binding (FIG. 7G).

### Example 8

### Effect of the Petunia hybrida Defensin PhD1a or Solanum Lycopersicum Defensin Tpp3 on the Permeabilisation of Human Cells In Vitro

### Example 8

### Introduction

To determine whether other defensins of the Solanaceae plant family were also able to permeabilise mammalian tumour cells in a similar manner to NaD1, the ability of the Petunia hybrida defensin PhD1A or Solanum lycopersicum (tomato) defensin TPP3 to permeabilise U937 cells was assessed using two approaches. The first used a bioluminescence assay to measure the release of intracellular ATP. 4×10⁴ U937 (human myelomonocytic tumour cell line) were treated with increasing concentrations of PhD1A or rTPP3 (0-20-μM) and ATP release measured at intervals of 30 seconds for a total of 30 min by determining absorbance at 562 nm. The second approach used flow cytometry to determine the uptake of the fluorescent dye propidium iodide (PI) (2 μg/mL) by U937 (4×10⁵/mL) following the treatment of cells with increasing concentrations of PhD1A (0 to 50 μM) or rTPP3 (0 to 40 μM) for 30 min.

### Example 8

### Results

U937 cells showed a release of ATP in a time-dependent and concentration-dependent manner when treated with native PhD1A (FIGS. 9B) or rTPP3 (FIG. 9D). Similar to NaD1, ATP was released from cells almost immediately upon exposure to PhD1A, or rTPP3. To further examine tumour cell permeabilisation by PhD1A or rTPP3, U937 cells were treated with increasing concentrations of PhD1A (0 to 50 μM) or rTPP3 (0 to 50 μM) for 30 min at 37° C. and then PI uptake measured by flow cytometry. As described for the release of ATP mediated by PhD1A or rTPP3, the uptake of PI by U937 cells increased with increasing concentrations of PhD1A or rTPP3. As shown in FIG. 9A, the number of PI⁺ U937 cells was ˜35% at 6.25 μM which increased to ˜90% PV at 50 μM. For TPP3, the number of PI⁺ U937 cells was ˜35% at 5 μM which increased to ˜90% PI⁺ at 40 μM (FIG. 9C).

### Example 9

### Comparison of the Permeabilisation Activity on U937 Cells by Solanaceous and Non-Solanacious Defensins/γ-Thionins

### Example 9

### Introduction

To assess the ability of solanaceous Class II defensins (NaD1, PhD1A and TPP3) to permeabilise tumour cells relative to non-solanaceous defensins and related γ-thionins (Dahlia merckii defensin Dm-AMP1, Hordeum vulgare gamma-thionin γ1-H, Zea mays gamma-thionin γ2-Z), the PI-uptake flow cytometry assay was utilised as described in Example 2 with the following modifications: 4×10⁵/mL U937 cells were incubated with 100 μM of each plant defensin/γ-thionin (NaD1, PhD1A, recombinant TPP3, recombinant γ1-H, and recombinant γ2-Z) for 60 min (in the absence of serum) followed by addition of 2 μg/mL PI. The percentage of PI⁺ cells was then determined by flow cytometry.

### Example 9

### Results

The three solanaceous Class II defensins, NaD1, PhD1A and rTPP3, all showed the ability to permeabilise U937 cells, as represented by the significantly increased number of PI⁺ cells compared to the cell only control; NaD1, PhD1A, and rTPP3 treatment at 10 μM resulted in 56.07±3.65%, 57.07±2.76%, and 49.97±2.93% PI⁺ cells (control 27.03±0.52). In contrast, no significant activity compared to the cell only control was observed for Dm-AMP1, γ1-H or γ2-z (FIG. 10).

### Example 10

### Permeabilisation Activity of the Petunia hybrida Defensin PhD1A or Solanum lycopersicum Defensin TPP3 in the Presence of Serum

### Example 10

### Introduction

To assess the ability of PhD1A or rTPP3 to permeabilise tumour cells in the presence of serum, the PI-uptake flow cytometry assay was utilised as described in Example 2 with the following modifications: 4×10⁵/mL U937 cells were incubated with 100 PhD1A or rTPP3 in the presence of increasing concentrations of foetal calf serum (FCS) (0 to 40%) for 60 min followed by addition of 2 μg/mL PI. The percentage of PI⁺ cells was then determined by flow cytometry.

### Example 10

### Results

Both PhD1A and rTPP3 retained the retained the ability to permeabilise U937 cells in the presence of serum, albeit at a reduced activity. For PhD1A, 40% PI⁺ cells were detected in the presence of 40% FCS compared to 90% PI⁺ cells at 0% FCS (FIG. 11A). Recombinant TPP3 appeared to show greater activity in serum than PhD1A, as exhibited by the retention of up to 70% activity in the presence of 5-40% FCS (FIG. 11B). It should be noted that the higher level of PI-positive cells at 0% FCS is a result of the complete absence of serum.

### Example 11

### Effect of the Native Tobacco (Nicotiana Suaveolens) Defensins NsD1, NsD2 and NsD3 on Permeabilisation of Human Tumour Cells In Vitro

### Example 11

### Introduction

To further investigate whether other class II defensins of the Solanaceae plant family are also able to permeabilise mammalian tumour cells in a similar manner to NaD1, and whether other class I defensins cannot, the ability of the Nicotiana suaveolens class II defensins NsD1 and NsD2, or the class I defensin NsD3, to permeabilise U937 cells was assessed in comparison to NaD1 using two approaches. The first used a bioluminescence assay to measure the release of intracellular ATP. 4×10⁴U937 were treated with 10 μM of each defensin and ATP release measured at intervals of 30 seconds for a total of 30 min by determining absorbance at 562 nm. The second approach used flow cytometry to determine the uptake of the fluorescent dye propidium iodide (PI) (2 μg/mL) by U937 (4×10⁵/mL) following the treatment of cells with 100 of each defensin for 30 min.

### Example 11

### Results

U937 cells showed a release of ATP in a time-dependent and concentration-dependent manner when treated with native NsD1 and NsD2 (FIG. 12A). Similar to NaD1, ATP was released from cells almost immediately upon exposure to NsD1 and NsD2. In contrast, native NsD3 did not mediate the release of ATP when compared to the cells only control (FIG. 12A). To further examine tumour cell permeabilisation by NsD1 and NsD2, versus NsD3, U937 cells were treated with 10 μM of each defensin for 30 min at 37° C. and then PI uptake measured by flow cytometry. NsD1 and NsD2 mediated the uptake of PI by U937 cells at similar levels to NaD1 (˜60% PI+ at 100), whereas NsD3 resulted in only low PI uptake (˜10% PI+ at 100) (FIG. 12B).

### Example 12

### Effect of Solanaceae Class II Defensins on Red Blood Cell Lysis

### Example 12

### Introduction

To determine if the inability of NaD1 to lyse human red blood cells (RBCs) was also conserved in other Solanaceae class II defensins, the ability of native NsD1, NsD2 and PhD1A to lyse RBCs was investigated by incubating 10⁷ RBCs with 10 μM or 30 μM of each defensin for 16 h at 37° C. and determining haemoglobin release by measuring absorbance at 412 nm.

### Example 12

### Results

Both NsD1 and PhD1A at 10 μM and 30 μM had no effect on RBC lysis when compared to the PBS only control. In comparison, NsD2 showed low hemolytic activity at 10 μM (˜17% lysis) and 30 μM (˜23% lysis) (FIG. 13).

### Example 13

### Cellular Lipid Binding Properties of Solanaceae Class I and II Defensins

### Example 13

### Introduction

Further investigation of the interaction of Solanaceae class I and class II defensins with cellular lipids was carried out by solid-state lipid binding assays using Echelon™ PIP Strips. The class I defensin NsD3, or the class II defensins NsD1, NsD2, PhD1a and rTPP3 (0.12 μM) were incubated overnight at 4° C. with lipid strips and binding detected with specific rabbit polyclonal antibodies to NaD2 or NaD1 (these antibodies cross-react with the class I or the class II defensins, respectively), followed by a HRP-conjugated donkey anti-rabbit antibody. Defensin binding was quantitated by densitometry on the developed lipid strips.

### Example 13

### Results

As described for NaD1, in general all of the class II defensins bound most strongly to the phosphoinositides PtdIns(PIP2) and (PIP3) including PtdIns(3,4)P2, PtdIns(3,5)P2, PtdIns(4,5)P2 and PtdIns(3,4,5)P3, but also showed binding to the PtdIns(PIP) including PtdIns(3)P, PtdIns(4)P, and PtdIns(5)P (FIGS. 14A, 14B, 14C and 14D). The exception was PhD1A, which also bound strongly to phosphatidic acid (FIG. 14E). The class I defensin NsD3 was also found to bind cellular lipids but with a specificity distinct to that of the class II defensins. In contrast to the class II defensins (with the exception of PhD1A), NsD3 showed strong binding to phosphatidic acid, and weak binding to the PtdIns(PIP), (PIP2) and (PIP3) (FIG. 14E). Collectively, these data suggest that Solanaceae class I and class II defensins bind cellular phospholipids with overlapping but different specificities, with class I defensins binding preferentially to phosphatidic acid and class II defensins to PtdIns (PIP), (PIP2) and (PIP3) (FIG. 7G).

### REFERENCES

Aluru et al. (1999) Plant Physiol 120: 633
Anaya-López et al. (2006) Biotechnol Lett 28: 1101-8.
Baker et al. (2005). Methods Enzymol 398: 540-554
Bensch et al. (1995) FEBS Let. 368:331-335
Bloch et al. (1991) FEBS Lett 279: 101-104
Bohlmann (1994) Crit. Rev Plant Sci 13: 1-16
Bohlmann and Apel (1991) Annu Rev Plant Physiol Plant Mol Biol 42: 227-240
Bonmatin et al. (1992) J Biomol NMR 2: 235-256
Brandstadter et al. (1996) Mol Gen Genet. 252: 146-154
Broekaert et al. (1992) Biochemistry 32: 4308-4314
Broekaert et al. (1995) Plant Physiol 108: 1353-1358
Broekaert et al. (1997) Crit. Rev Plant Sci 16: 297-323
Bruix et al. (1993) Biochemistry 32: 715-724
Cammue et al. (1992) J Biol Chem 267: 2228-2233
Catanzariti et al. (2004) Protein Sci 13: 1331-1339
Chen et al. (2002) J Agric Food Chem 50: 7258-63
Colilla et al. (1990) FEBS Lett 270: 191-4
Cornet et al., (1995) Structure 3: 435-448
Craik et al. (1999) J Mol Biol 294: 1327-1336
Craik (2001) Toxicon 39: 1809-1813
Craik et al. (2004) Curr Prot Pept Sci 5: 297-315
Da Silva et al. (2003) Protein Sci 12: 438-446
de Zélicourt et al. (2007) Planta 226: 591-600
Diamond et al. (1996) Proc Natl Acad Sci USA 93: 5156-5160
Fehlbaum et al. (1994) J Biol Chem 269: 33159-33163
Ganz et al. (1985) J Clin Invest 76: 1427-1435
Gu et al. (1992) Mol Gen Genet. 234: 89-96
Gustafson et al. (1994) J Am Chem Soc 116: 9337-9338
Hancock and Lehrer (1998) Trends Biotech 16: 82-88
Hanzawa et al. (1990) FEBS Lett 269: 413-420
Harlow and Lane (1988) Antibodies: A Laboratory Manual. Cold Spring Harbor Laboratory Press, Cold Spring Harbor, N. Y.
Harwig et al. (1992) Blood 79: 1532-1537
Janssen et al. (2003) Biochemistry 42: 8214-8222
Jennings et al. (2001) Proc Natl Acad Sci U.S.A. 98: 10614-10619
Kader (1996) Annu Rev Plant Physiol Plant Mol Biol 47: 627-654
Kader (1997) Trends Plant Sci 2: 66-70
Komori et al. (1997) Plant Physiol 115: 314
Kushmerick et al. (1998) FEBS Lett 440: 302-306
Lamberty et al. (1999) J Biol Chem 274: 9320-9326
Lamberty et al. (2001) Biochemistry 40: 11995-12003
Landon et al. (1997) Protein Sci 6: 1878-1884
Lay and Anderson (2005) Curr Protein Pept Sci 6: 85-101
Lay et al. (2003a) Plant Physiol. 131: 1283-1293
Lay et al. (2003b) J Mol Biol 325: 175-188
Lehrer and Ganz (2002) Curr Opin Immunol 14: 96-102
Lin et al. (2009) Biosci Rep 30: 101-109
Lobo et al. (2007) Biochemistry 46: 987-96
Loenza-Angeles et al. (2008) 30: 1713-1719
Ma et al. (2009) Peptides 30: 2089-2094
Mader and Hoskin (2006) Expert Opin Investig Drugs 15: 933-46. Review
Marcus et al. (1997) Eur J Biochem 244: 743-749
McManus et al. (1999) J Mol Biol 293: 629-638
Melo et al. (2002) Proteins 48: 311-319
Mendez et al. (1990) Eur J Biochem 194: 533-539
Mendez et al. (1996) Eur J Biochem 239: 67-73
Milligan et al. (1995) Plant Mol Biol 28: 691-711
Mirouze et al. (2006) Plant J 47: 329-342
Moreno et al. (1994) Eur J Biochem 223: 135-139
Ngai and Ng (2005) Biochem Cell Biol 83: 212-20
Osborn et al. (1995) FEBS Lett 368: 257-262
Patel et al. (1998) Biochemistry 37: 983-990
Pelegrini and Franco (2005) Int J Biochem Cell Biol 37: 2239-53
Ramamoorthy et al. (2007) Mol Microbiol 66: 771-786
Russell et al. (1996) Infect Immun 64: 1565-1568
Segura et al. (1998) FEBS Lett 435: 159-162
Selsted et al. (1985) J Clin Invest 76: 1436-1439
Selsted et al. (1993) J Biol Chem 268: 6641-6648
Tailor et al. (1997) J Biol Chem 272: 24480-24487
Tam et al. (1999) Proc Natl Acad Sci U.S.A. 96: 8913-8918
Tang and Selsted (1993) J Biol Chem 268: 6649-6653
Tang et al. (1999a) Science 286: 498-502
Tang et al. (1999b) Infect Immun 67: 6139-6144
Tarver et al. (1998) Infect Immun 66: 1045-1056
Terras et al. (1992) J Biol Chem 267: 15301-15309
Terras et al. (1993) FEBS Lett 316: 233-240
Terras et al. (1995) Plant Cell 7: 573-588
Thomma et al. (2003) Curr Drug Targets—Infect. Dis. 3: 1-8
Trabi et al. (2001) Biochemistry 40: 4211-4221
van der Weerden (2008) J Biol Chem 283: 14445-14452
Wijaya et al. (2000) Plant Sci 159: 243-255
Wong and Ng (2005) Int J Biochem Cell Biol 37: 1626-32
Yamada et al. (1997) Plant Physiol 115: 314
Zhang and Lewis (1997) FEMS Microbiol Lett 149: 59-64
Zhang et al. (1997) Cereal Chem 74: 119-122

1. A method for preventing or treating a proliferative disease, wherein the method comprises administering to a subject a therapeutically effective amount of a Solanaceous Class II plant defensin, thereby preventing or treating the proliferative disease.

2. The method of claim 1, wherein the plant defensin is derived from Nicotiana alata, Nicotiana suaveolens, Petunia hybrida or Solanum lycopersicum.

3. The method of claim 1, wherein the plant defensin is selected from the group consisting of NaD1, NsD1, NsD2, PhD1A and TPP3.

4. The method of claim 1, wherein the plant defensin comprises an amino acid sequence selected from the group consisting of SEQ ID NO: 1, SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, or SEQ ID NO: 18, SEQ ID NO: 20, SEQ ID NO: 22, SEQ ID NO: 24 and SEQ ID NO: 26.

5. The method of claim 1, wherein the plant defensin comprises a functional fragment of a plant defensin comprising an amino acid sequence selected from the group consisting of SEQ ID NO: 1, SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, or SEQ ID NO: 18, SEQ ID NO: 20, SEQ ID NO: 22, SEQ ID NO: 24 and SEQ ID NO: 26.

6. The method of claim 1, wherein the proliferative disease is cancer.

7. The method of claim 6, wherein the cancer is selected from the group consisting of basal cell carcinoma, bone cancer, bowel cancer, brain cancer, breast cancer, cervical cancer, leukemia, liver cancer, lung cancer, lymphoma, melanoma, ovarian cancer, pancreatic cancer, prostate cancer and thyroid cancer.

8. A kit for use in preventing or treating a proliferative disease, wherein the kit comprises a Solanaceous Class II plant defensin and instructions for administration in the treatment or prevention of a proliferative disease.

9. A method for screening for cytotoxicity of a plant defensin against mammalian tumour cells, wherein the method comprises contacting the plant defensin with a mammalian cell line, and assaying for cytoxicity against the mammalian cell line due to contact with the plant defensin.

10. A Solanaceous Class II plant defensin with reduced haemolytic activity.

11. The plant defensin of claim 10 comprising at least one alanine residue at or near the N-terminal of the defensin.

12. A method of making a Solanaceous Class II plant defensin with reduced haemolytic activity comprising adding one or more codons encoding Alanine to a nucleic acid encoding a Solanaceous Class II plant defensin.


SEQUENCE LISTING (27 sequences)

SEQ ID NO: 1 - protein, 50 aa, Artificial
  Synthetic polypeptide
XXXCXXXXXXXXXXCXXXXXCXXXCXXXXXXXXXCXXXXXXXXCXCXXXC

SEQ ID NO: 2 - protein, 105 aa, Nicotiana alata
MARSLCFMAFAILAMMLFVAYEVQARECKTESNTFPGICITKPPCRKACISEKFTDGHCSKILRRCLCTKPCVFDEKMTKTGAEILAEEAKTLAAALLEEEIMDN

SEQ ID NO: 3 - DNA, 318 bp, Nicotiana alata
atggctcgctccttgtgcttcatggcatttgctatcttggcaatgatgctctttgttgcctatgaggtgcaagctagagaatgcaaaacagaaagcaacacatttcctggaatatgcattaccaaaccaccatgcagaaaagcttgtatcagtgagaaatttactgatggtcattgtagcaaaatcctcagaaggtgcctatgtactaagccatgtgtgtttgatgagaagatgactaaaacaggagctgaaattttggctgaggaagcaaaaactttggctgcagctttgcttgaagaagagataatggataactaa

SEQ ID NO: 4 - protein, 47 aa, Nicotiana alata
RECKTESNTFPGICITKPPCRKACISEKFTDGHCSKILRRCLCTKPC

SEQ ID NO: 5 - DNA, 144 bp, Nicotiana alata
agagaatgcaaaacagaaagcaacacatttcctggaatatgcattaccaaaccaccatgcagaaaagcttgtatcagtgagaaatttactgatggtcattgtagcaaaatcctcagaaggtgcctatgtactaagccatgttaa

SEQ ID NO: 6 - protein, 48 aa, Nicotiana alata
ARECKTESNTFPGICITKPPCRKACISEKFTDGHCSKILRRCLCTKPC

SEQ ID NO: 7 - DNA, 147 bp, Nicotiana alata
gctagagaatgcaaaacagaaagcaacacatttcctggaatatgcattaccaaaccaccatgcagaaaagcttgtatcagtgagaaatttactgatggtcattgtagcaaaatcctcagaaggtgcctatgtactaagccatgttaa

SEQ ID NO: 8 - protein, 105 aa, Solanum lycopersicum
MARSIFFMAFLVLAMMLFVTYEVEAQQICKAPSQTFPGLCFMDSSCRKYCIKEKFTGGHCSKLQRKCLCTKPCVFDKISSEVKATLGEEAKTLSEVVLEEEIMME

SEQ ID NO: 9 - DNA, 318 bp, Solanum lycopersicum
atggctcgttccattttcttcatggcatttttggtcttggcaatgatgctctttgttacctatgaggtagaagctcagcaaatttgcaaagcaccaagccaaactttcccaggattatgttttatggactcatcatgtagaaaatattgtatcaaagagaaatttactggtggacattgtagcaaactccaaaggaagtgtctatgcactaagccatgtgtatttgacaaaatctcaagtgaagttaaagcaactttgggtgaggaagcaaaaactctaagtgaagttgtgcttgaagaagagattatgatggagtaa

SEQ ID NO: 10 - protein, 48 aa, Solanum lycopersicum
QQICKAPSQTFPGLCFMDSSCRKYCIKEKFTGGHCSKLQRKCLCTKPC

SEQ ID NO: 11 - DNA, 147 bp, Solanum lycopersicum
cagcaaatttgcaaagcaccaagccaaactttcccaggattatgttttatggactcatcatgtagaaaatattgtatcaaagagaaatttactggtggacattgtagcaaactccaaaggaagtgtctatgcactaagccatgttaa

SEQ ID NO: 12 - protein, 49 aa, Solanum lycopersicum
AQQICKAPSQTFPGLCFMDSSCRKYCIKEKFTGGHCSKLQRKCLCTKPC

SEQ ID NO: 13 - DNA, 150 bp, Solanum lycopersicum
gctcagcaaatttgcaaagcaccaagccaaactttcccaggattatgttttatggactcatcatgtagaaaatattgtatcaaagagaaatttactggtggacattgtagcaaactccaaaggaagtgtctatgcactaagccatgttaa

SEQ ID NO: 14 - protein, 101 aa, Petunia hybrida
MARSICFFAVATLALMLFAAYEAEAATCKAECPTWDGICINKGPCVKCCKAQPEKFTDGHCSKVLRRCLCTKPCATEEATATLANEVKTMAEALVEEDMME

SEQ ID NO: 15 - DNA, 306 bp, Petunia hybrida
atggctcgctccatctgtttcttcgcagttgctacactggcattgatgctctttgctgcctatgaggcggaagcggcaacttgcaaggctgaatgcccaacttgggatggaatatgtataaataaaggcccatgtgtaaaatgttgcaaagcacaaccagaaaaattcacagacgggcactgcagtaaagtactccgaagatgcctatgcactaagccgtgtgcaactgaagaggcaactgcaactttggctaacgaggtaaagactatggctgaagctttggtcgaagaagatatgatggaataa

SEQ ID NO: 16 - protein, 101 aa, Petunia hybrida
MARSICFFAIAMLALMLFVSYEAEAATCKAECPTWDGICINKGPCVKCCKAQPEKFTDGHCSKVLRRCLCTKPCATEEATATLANEVKTMAEALVEEDMME

SEQ ID NO: 17 - DNA, 306 bp, Petunia hybrida
atggctcgctccatctgtttcttcgcaattgccatgctagcattgatgctcttcgtttcgtatgaggcggaagcggcaacttgcaaggctgaatgcccaacttgggatggaatatgtataaataaaggcccatgtgtaaaatgttgcaaagcacaaccagaaaaattcacagacgggcactgcagtaaagtactccgaagatgcctatgcactaagccgtgtgcaactgaagaggcaactgcaactttggctaacgaggtaaagactatggctgaagctttggtcgaagaagatatgatggagtaa

SEQ ID NO: 18 - protein, 49 aa, Petunia hybrida
ATCKAECPTWDGICINKGPCVKCCKAQPEKFTDGHCSKVLRRCLCTKPC

SEQ ID NO: 19 - DNA, 147 bp, Petunia hybrida
gcaacttgcaaggctgaatgcccaacttgggatggaatatgtataaataaaggcccatgtgtaaaatgttgcaaagcacaaccagaaaaattcacagacgggcactgcagtaaagtactccgaagatgcctatgcactaagccgtgt

SEQ ID NO: 20 - protein, 105 aa, Nicotiana suaveolens
MARSLCFMAFAILAVMLFVAYDVEAKDCKRESNTFPGICITKPPCRKACIREKFTDGHCSKILRRCLCTKPCVFDEKMIETGAETLAEEAKTLAAALLEEEIMDN

SEQ ID NO: 21 - DNA, 318 bp, Nicotiana suaveolens
atggctcgctccttgtgcttcatggcatttgctatcttggcagtgatgctctttgttgcctatgatgtggaagctaaagattgcaaaagagaaagcaatacattccctggaatatgcattaccaaaccaccatgcagaaaagcttgtatccgtgagaaatttactgatggtcattgtagcaaaatcctcagaagatgtctatgcactaagccatgtgtgtttgatgagaagatgatcgaaacaggagctgaaactttagctgaggaagcaaaaactttggctgcagctttgcttgaagaagagataatggataactga

SEQ ID NO: 22 - protein, 47 aa, Nicotiana suaveolens
KDCKRESNTFPGICITKPPCRKACIREKFTDGHCSKILRRCLCTKPC

SEQ ID NO: 23 - DNA, 141 bp, Nicotiana suaveolens
aaagattgcaaaagagaaagcaatacattccctggaatatgcattaccaaaccaccatgcagaaaagcttgtatccgtgagaaatttactgatggtcattgtagcaaaatcctcagaagatgtctatgcactaagccatgt

SEQ ID NO: 24 - protein, 105 aa, Nicotiana suaveolens
MARSLCFMAFAILAMMLFVAYDVEAKDCKRESNTFPGICITKLPCRRACISEKFADGHCSKILRRCLCTKPCMFDEKMIKTGAETLAEEAKTLAAALLEEEIMDN

SEQ ID NO: 25 - DNA, 318 bp, Nicotiana suaveolens
atggctcgctccttgtgcttcatggcatttgctatcttggcaatgatgctctttgttgcctatgatgttgaagctaaagattgcaaaagagaaagcaatacattccctggaatatgcattaccaaactaccatgcagaagagcttgtatcagtgagaaatttgctgatggtcattgtagcaaaatcctcagaaggtgtctatgcactaagccatgtatgtttgatgagaagatgatcaaaacaggagctgaaactttagctgaggaagcaaaaactttggctgcagctttgcttgaagaagagataatggataactga

SEQ ID NO: 26 - protein, 47 aa, Nicotiana suaveolens
KDCKRESNTFPGICITKLPCRRACISEKFADGHCSKILRRCLCTKPC

SEQ ID NO: 27 - DNA, 141 bp, Nicotiana suaveolens
aaagattgcaaaagagaaagcaatacattccctggaatatgcattaccaaactaccatgcagaagagcttgtatcagtgagaaatttgctgatggtcattgtagcaaaatcctcagaaggtgtctatgcactaagccatgt